(19) United States
(12) Patent Application Publication
(10) Pub. No.: US 2019/0350852 A1
(43) Pub. Date: Nov. 21, 2019

(54) MATERIALS AND METHODS FOR ADMINISTRATION TO NASAL CAVITY

(71) Applicant: MACULUS THERAPEUTIX LLC, Mountain View, CA (US)

(72) Inventor: Syed Hasan ASKARI, San Jose, CA (US)

(21) Appl. No.: 16/531,245

(22) Filed: Aug. 5, 2019

Related U.S. Application Data
(63) Continuation of application No. PCT/US2018/061668, filed on Nov. 16, 2018.
(60) Provisional application No. 62/587,156, filed on Nov. 16, 2017.

(51) Int. Cl. A61K 9/06; A61K 9/00; A61K 47/34; A61P 17/02

(52) U.S. Cl. CPC A61K 9/06; A61P 17/02; A61K 47/34; A61K 9/0043

(57) ABSTRACT

The present disclosure provides compositions, devices, systems, methods, and kits for treatment of nasal conditions. The compositions and methods of the present disclosure may include a biodegradable polymer formed by, for example, a Michael addition reaction between a thiol moiety and an acrylate moiety.

### CROSS-REFERENCE

This application is a continuation of International Application No. PCT/US2018/061668, filed Nov. 16, 2018, which claims the benefit of U.S. Provisional Application No. 62/587,156, filed Nov. 16, 2017; the disclosure of each of the prior applications is considered part of and is incorporated by reference in the disclosure of this application.

### BACKGROUND

Rhinosinusitis is an inflammatory condition of the nasal cavity with a high global prevalence. Rhinosinusitis is characterized by nasal congestion and/or obstruction, nasal discharge, postnasal drainage, swelling of the nose and surrounding areas, facial pain and/or pressure, fever, and loss of smell affects millions of Americans every year. Rhinosinusitis may be acute or chronic. While acute rhinosinusitis may persist for weeks, chronic rhinosinusitis (CRS) can extend for months. Both acute and chronic rhinosinusitis can negatively affect quality of life and normal functioning. CRS may be particularly challenging as it is also associated with co-morbid conditions including asthma and eczema and can lead to tissue damage.

CRS affects nearly 10% of Americans. Approximately 25-30% of individuals with CRS develop nasal polyps. Nasal polyps may obstruct sinonasal passages. Though antibiotic, steroid, and/or immuno therapies may be used to treat the underlying causes of these growths, surgery (e.g., Functional Endoscopic Sinus Surgery) is often required to remove nasal polyps and restore sinonasal passages. However, surgery involves considerable risk to both nasal passages and other facial organs, and nasal polyps have a tendency to regrow, necessitating additional surgical procedures. Further, nasal polyp surgery can result in significant bleeding, for which existing treatments are inadequate. Such treatments typically include absorbent packing materials that mechanically stop blood flow. These treatments are often uncomfortable for patients, block air passages, and can result in infection. Moreover, patients generally have to return to a surgeon's office for removal of the packing material, which removal may cause additional bleeding.

### SUMMARY

The present disclosure provides compositions, methods, and kits for use in treating nasal conditions and/or promoting hemostasis (e.g., in nasal passages). The compositions provided herein may comprise a biodegradable polymer that may be generated by, for example, a Michael addition reaction between a thiol moiety and an acrylate moiety. Such components may be combined immediately before application of the composition to a target area of an individual. The biodegradable polymers provided herein may degrade over, for example, 2-8 weeks and may promote hemostasis.

In an aspect, the present disclosure provides a biodegradable polymer generated by combining: (a) at least one first compound comprising two or more thiol moieties and (i) two or more ethoxy moieties or (ii) two or more carboxyl moieties; and (b) at least one second compound comprising one or more acrylate moieties.

In some embodiments, said at least one first compound comprises three or more thiol moieties. In some embodiments, said at least one first compound comprises three or more carboxyl moieties. In some embodiments, said at least one first compound comprises three or more thiol moieties, wherein said thiol moieties are each represented by the formula:

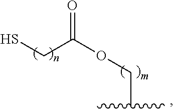

wherein n and m are for each thiol moiety are each independently selected from the group consisting of 1, 2, and 3. In some embodiments, said at least one first compound comprises at least six or more of said thiol moieties. In some embodiments, said at least one first compound comprises eight of said thiol moieties. In some embodiments, each n is 2 and each m is 1. In some embodiments, said at least one first compound is represented by the formula:

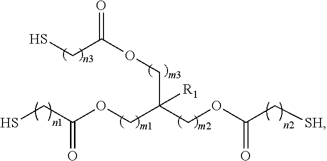

wherein R₁ is selected from the group consisting of H and C₁-C₆ alkyl; n₁, n₂, and n₃ are each independently selected from the group consisting of 1, 2, and 3; and m₁, m₂, and m₃ are each independently selected from the group consisting of 1, 2, and 3. In some embodiments, R₁ is C₁ alkyl. In some embodiments, n₁, n₂, and n₃ are each 2. In some embodiments, m₁, m₂, and m₃ are each 1. In some embodiments, said at least one first compound is represented by the formula:

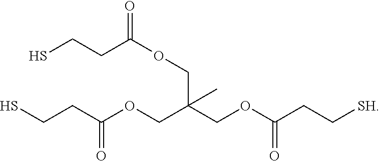 [SMILES: CC(COC(=O)CCS)(COC(=O)CCS)COC(=O)CCS]

In some embodiments, said at least one first compound comprises three or more ethoxy moieties. In some embodiments, said at least one first compound comprises three or more thiol moieties, wherein said thiol moieties are each represented by the formula:

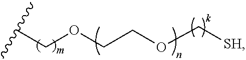

wherein k and m for each thiol moiety are each independently selected from the group consisting of 1, 2, and 3 and wherein n is selected from the group consisting of 1-500. In some embodiments, said at least one compound comprises at least six of said thiol moieties. In some embodiments, said at least one compound comprises eight of said thiol moieties. In some embodiments, each m is 1. In some embodiments, each k is 2. In some embodiments, said at least one first compound is represented by the formula:

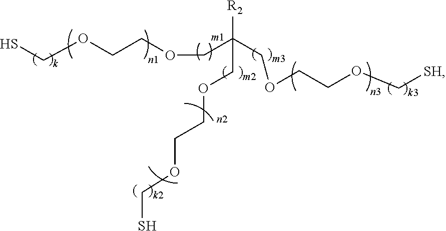

where R₂ is selected from H, C₁₋₆ alkyl, and

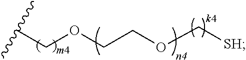

n₁, n₂, n₃, and n₄ (if present) are each independently selected from the group consisting of 1-500; m₁, m₂, m₃, and m₄ (if present) are each independently selected from the group consisting of 1, 2, and 3; and k₁, k₂, k₃, and k₄ (if present) are each independently selected from the group consisting of 1, 2, and 3. In some embodiments, said at least one first compound is represented by the formula:

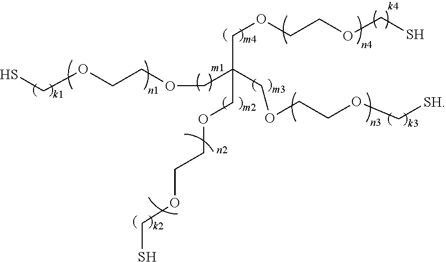

In some embodiments, m₁, m₂, m₃, and m₄ are each 1. In some embodiments, k₁, k₂, k₃, and k₄ are each 2. In some embodiments, said at least one first compound is represented by the formula:

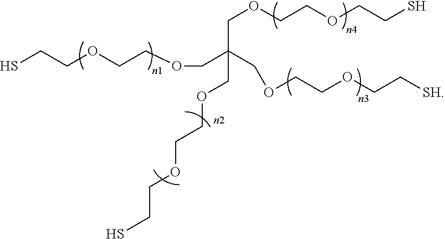

In some embodiments, said at least one second compound comprises three or more acrylate moieties. In some embodiments, said acrylate moieties are each represented by the formula:

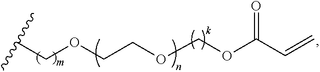

wherein n for each acrylate moiety is selected from the group consisting of 1-500, such as 1-20; and m and k for each acrylate moiety are each independently selected from the group consisting of 1, 2, and 3. In some embodiments, said at least one second compound comprises at least six of said acrylate moieties. In some embodiments, said at least one second compound comprises eight of said acrylate moieties. In some embodiments, each m is 1. In some embodiments, each k is 2.

In some embodiments, said at least one second compound comprises one or polyethylene glycol moieties.

In some embodiments, said at least one second compound is represented by the formula:

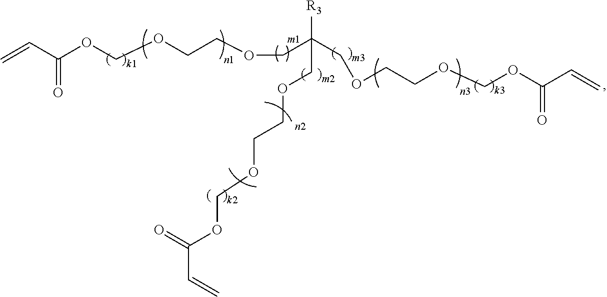

wherein R₃ is selected from the group consisting of H, C₁-C₆ alkyl, and

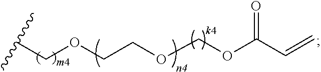

n₁, n₂, n₃, and n₄ (if present) are each independently selected from the group consisting of 1-500; m₁, m₂, m₃, and m₄ (if present) are each independently selected from the group consisting of 1, 2, and 3; and k₁, k₂, k₃, and k₄ (if present) are each independently selected from the group consisting of 1, 2, and 3. In some embodiments, said at least one second compound is represented by the formula:

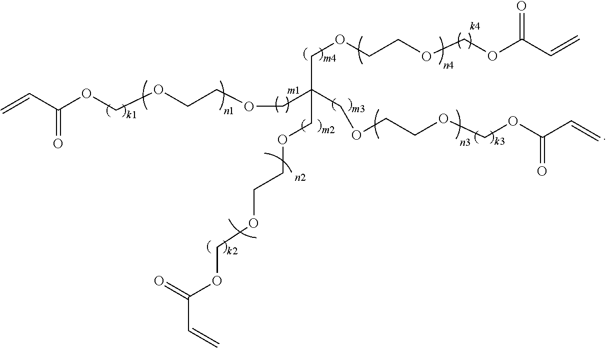

In some embodiments, m₁, m₂, m₃, and m₄ are each 1. In some embodiments, k₁, k₂, k₃, and k₄ are each 2. In some embodiments, said at least one second compound is represented by the formula:

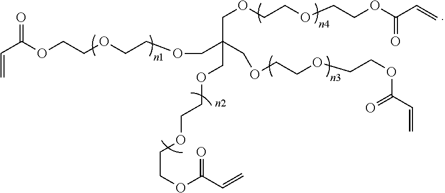

In some embodiments, said acrylate moieties are each represented by the formula:

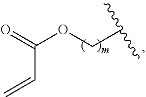

wherein m of each moiety is independently selected from the group consisting of 1, 2, and 3. In some embodiments, each m is 1. In some embodiments, said at least one second compound is represented by the formula:

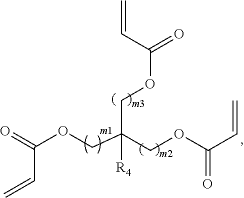

wherein R₄ is selected from the group consisting of H, C₁-C₆ alkyl, and C₁-C₆ hydroxyalkyl; and m₁, m₂, and m₃ and are each independently selected from the group consisting of 1, 2, and 3. In some embodiments, R₄ is selected from C₁-C₆ hydroxyalkyl. In some embodiments, R₄ is C₁ hydroxyalkyl. In some embodiments, m₁, m₂, and m₃ and are each 1. In some embodiments, said at least one second compound is represented by the formula:

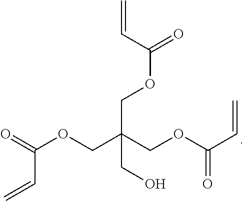 [SMILES: C=CC(=O)OCC(CO)(COC(=O)C=C)COC(=O)C=C]

In some embodiments, said at least one first compound is represented by the formula:

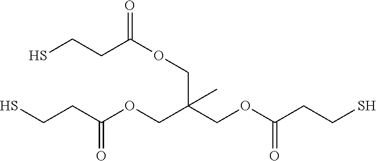 [SMILES: CC(COC(=O)CCS)(COC(=O)CCS)COC(=O)CCS]

and said at least one second compound is represented by the formula:

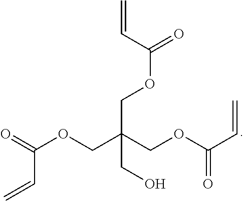 [SMILES: C=CC(=O)OCC(CO)(COC(=O)C=C)COC(=O)C=C]

In some embodiments, said at least one first compound is represented by the formula:

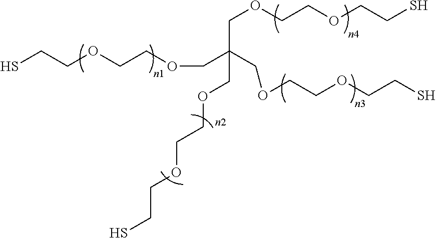

and said at least one second compound is represented by the formula:

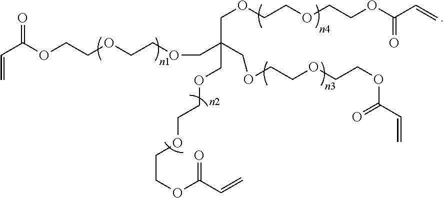

In some embodiments, said at least one first compound and said at least one second compound are combined at a pH between 7-9. In some embodiments, generating said biodegradable polymer comprises combining a first solution comprising said at least one first compound and a second solution comprising said at least one second compound. In some embodiments, generating said biodegradable polymer comprises combining said at least one first compound and said at least one second compound in the presence of acetonitrile. In some embodiments, said biodegradable polymer has a durometer of greater than about 40. In some embodiments, said biodegradable polymer degrades between about 2 and about 8 weeks.

In some embodiments, said biodegradable polymer comprises a therapeutic agent selected from the group consisting of a corticosteroid, a decongestant, an immunotherapeutic agent, an adrenocorticoid, an analgesic agent, an analgesic adjunct, an analgesic-anesthetic, an anesthetic, an antibiotic, an antibacterial agent, an anti-infective agent, an antibiotic therapy adjunct, an antidote, an anti-emetic agent, an anti-fungal agent, an anti-inflammatory agent, an anti-vertigo agent, an anti- viral agent, a biological response modifier, a cytotoxic agent, a diagnostic aid, an immunizing agent, an immunomodulator, proteins, and peptides.

In another aspect, the present disclosure provides a composition comprising any of the biodegradable polymers provided herein.

In a further aspect, the present disclosure provides a method of treating a nasal condition in an individual in need thereof, comprising administering to said individual a composition as provided herein.

In some embodiments, said nasal condition is selected from the group consisting of sinusitis, nasal polyps, nasal obstruction, rhinitis, bacterial infection, nosebleed, and nasal tumors. In some embodiments, said nasal condition is sinusitis. In some embodiments, said sinusitis is selected from the group consisting of acute sinusitis, recurrent sinusitis, and chronic sinusitis. In some embodiments, said individual has had surgery to remove one or more nasal polyps. In some embodiments, said composition is configured to be applied as a liquid that hardens upon application to the nasal cavity.

In yet another aspect, the present disclosure provides a method of promoting hemostasis in an individual in need thereof, comprising administering to said individual a composition as provided herein.

In some embodiments, said individual has had surgery to remove one or more nasal polyps. In some embodiments, said composition is configured to be applied as a liquid that hardens upon application to the nasal cavity.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a comparison of a polymer composition 1 and 20 days after generation.

FIG. 2 graphically depicts that polymer compositions significantly reduced bleeding time after cutting a mouse tail.

FIG. 3 shows that polymer compositions promoted hemostasis for a severed mouse tail.

FIGS. 4A-4D show that polymer compositions promoted hemostasis for severed tails for several mice.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### Definitions

The term “aperture” refers to a hole or opening.

The term “biocompatible” or “biologically compatible,” as used interchangeably herein, refer to materials that are, with any metabolites or degradation products thereof, generally nontoxic to the recipient, and cause no significant adverse effects to the recipient. Generally speaking, biocompatible materials are materials which do not elicit a significant inflammatory or immune response when administered to a patient. In some embodiments a biocompatible material elicits no detectable change in one or more biomarkers indicative of an immune response. In some embodiments, a biocompatible material elicits no greater than a 10% change, no greater than a 20% change, or no greater than a 40% change in one or more biomarkers indicative of an immune response.

The terms “biodegradable”, “bioerodable”, “biologically absorbable,” and “bioresorbable,” in reference to polymers and coatings, are used interchangeably and refer to polymers and coatings that are capable of being completely or substantially completely degraded, dissolved, and/or eroded over time when exposed to bodily fluids such as blood and can be gradually resorbed, absorbed and/or eliminated by the body, or that can be degraded into fragments that can pass through the kidney membrane of a human (e.g., fragments having a molecular weight of about 40,000 Daltons (40 kDa) or less). The process of breaking down and eventual absorption and elimination of the polymer or coating can be caused by, e.g., hydrolysis, metabolic processes, oxidation, enzymatic processes, bulk or surface erosion, and the like. Conversely, a “biostable” polymer or coating refers to a polymer or coating that is not biodegradable.

The term “co-polymer” as used herein refers to a variety of polymer wherein non-identical repeating units are present. A copolymer can be regular or random in the sequence defined by the more than one type of repeating unit. Some types of copolymers are random copolymers, graft copolymers and block copolymers.

The term “durometer” as used herein refers to the hardness of a polymer measured using the Shore A, one second scale for plastics or ASTM Method 2240.

The term “effective amount” as used herein with reference to a drug, compound, or pharmaceutical composition is an amount sufficient to effect beneficial or desired results including modulation of clinical manifestations or symptoms such as a decrease in, for example, erythema, edema, pruritus, or changes in general clinical results such as congestion, nasal obstruction, nasal discharge, post-nasal drip, polyp size, polyp growth rate, polyp number, facial tenderness, results of measures of sensory function, fever, loss of appetite, vomiting, and halitosis, resolution of sinusitis, eradication of pathogen, and decreased relapse rates; or increasing the quality of life of those suffering from the disease (for example, increasing physical functioning, decreasing bodily pain, increasing general health, increasing vitality, increasing social functioning), decreasing the dose of other medications, e.g. palliative care medications or other medications, required to treat the disease, delaying the progression of the disease, decreasing time required for resolution of infection and/or symptoms, and/or prolonging survival of patients. An effective amount can be administered in one or more administrations. In some embodiments, an effective amount of drug, compound, or pharmaceutical composition is an amount sufficient to decrease clinical manifestations of otitis media.

The term “inhibit,” “inhibiting,” or “inhibition” as used herein refers to a decrease in activity, response, condition, disease, or other biological parameter. This can include, but is not limited to, the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “incision” refers to a cut or penetration. In some embodiments, an incision extends beyond the dermal layer of a patient's skin. An incision can be made with, for example, a knife, needle, blade, lancet, scalpel, laser, or other mechanism.

The term “Michael reaction” or “Michael addition” refers to a chemical reaction between a nucleophile (the “Michael donor”) and an activated olefin or alkyne (the “Michael acceptor”) in which the nucleophile adds across a carbon-carbon multiple bond, resulting in a “Michael adduct.” The Michael donor can be a carbon-nucleophile or a hetero-atomic nucleophile.

The term “microorganism” or “microbe” as used herein refers to bacteria, yeast, mold, fungi, protozoa, mycoplasma, as well as viruses (including lipid enveloped RNA and DNA viruses).

The term “modulus” as used herein refers to the ratio of a component of stress or force per unit area applied to a material divided by the strain along an axis of applied force that results from the applied force. The modulus or the stiffness typically is the initial slope of a stress-strain curve at low strain in the linear region. For example, a material has both a tensile and a compressive modulus.

The term “molecular weight” as used herein with reference to a polymer refers to a number average molecular weight, a weight average molecular weight, a peak average molecular weight, a Z average molecular weight, a viscosity average molecular weight, or a melt index of the polymer. The number average molecular weight Mₙ is defined as

$$\frac{\sum {N}_{i} {M}_{i}}{\sum {N}_{i}}$$

where Mᵢ is the molecular weight of a chain and Nᵢ is the number of chains of that molecular weight. The weight average molecular weight M_{w} is defined as

$$\frac{\sum {N}_{i} {M}_{i}^{2}}{\sum {N}_{i} {M}_{i}} .$$

The Z average molar mass M_{z} is defined as

$$\frac{\sum {N}_{i} {M}_{i}^{3}}{\sum {N}_{i} {M}_{i}^{2}} .$$

The viscosity average molecular weight Mᵥ is defined as

$${[ \frac{\sum {N}_{i} {M}_{i}^{1 + a}}{\sum {N}_{i} {M}_{i}} ]}^{1 / a} .$$

The term “patency” or “patent” as used herein refers to the quality or state of being open or unobstructed. It can be expressed in a percentage.

The term “patency maintaining agent” refers to any agent that promotes patency over a suitable period of time. Patency can be measured over a period of days, weeks, months or years.

The term “polymer” as used herein refers to a macromolecule made up of a series of at least about 10, and preferably more, repeating units. In some embodiments, a polymer has at least 10, 20, 30, 40, 50 100, 200, 300, 400, 500, 600, 700, 800, 900, 1000, or more repeating units. Repeating units are linked together by, for example, covalent chemical linkages or non-covalent linkages. In some embodiments, a polymer has a number average molecular weight of more than about 500 g/mol. Polymer types include, but are not limited to, homopolymers (only one type of repeat unit), copolymers (two or more types of repeat units), a blend of homopolymers, a blend of copolymers, and a blend of one or more homopolymers and one or more copolymers.

The term “prevent,” “preventing,” or “prevention” as used herein does not require absolute forestalling of the condition or disease but can also include a reduction in the onset or severity of the disease or condition or inhibition of one or more symptoms of the disease or disorder.

The term “pro-drug” refers to any compound that is converted to a more pharmacologically active compound under physiological conditions (i.e., in vivo). A common method for making a pro-drug is to select moieties that are hydrolyzed under physiological conditions to provide the desired biologically active drug.

The term “sustained-release composition” refers to a composition that comprises at least one therapeutic agent (i.e., a drug, pro-drug, or co-drug) and which is capable of releasing the therapeutic agent at a steady rate over a prolonged period of time ranging from a week to a year or more, upon administration to an individual in need thereof.

The term “treating” as used herein refers to means ameliorating, improving or remedying a disease, disorder, or symptom of a disease or condition.

### Polymer Materials

The present disclosure provides compositions, methods, materials, and kits useful for treating conditions of the ear, nose, and/or throat (e.g., nasal conditions) and/or promoting hemostasis (e.g., in a nasal passage). The compositions provided herein comprise a polymer (e.g., a biodegradable polymer) that can be provided to a treatment area (e.g., manually, using an applicator, or by spraying). The polymer gels upon application to the treatment area. In some embodiments, the compositions provided herein are capable of promoting hemostasis while keeping bodily passages (e.g., nasal passages) open without imparting excess pressure, unlike other treatments that utilize absorbent packing material. Alternatively, the polymers of the present disclosure can be provided to a treatment area via an absorbant material such as a super absorbing gel pad capable of absorbing fluid quickly, temporarily drying the surface of the treatment area, and providing hemostasis. Accordingly, in some embodiments, the compositions and materials provided herein both protect wounds from infection and enable observation of wound healing. In some embodiments, the compositions provided herein include bioabsorbable materials that are absorbed by the body over 2-8 weeks, such as over 2-4 weeks.

Polymers (e.g., biodegradable polymers) of the present disclosure can be prepared using any useful starting materials (e.g., polymerizable materials), and any useful amount or ratio thereof. For example, a first material and a second material are used to generate a polymer. In some embodiments, equal amounts of the first and second materials are used to generate the polymer. In other embodiments, a greater amount of the first material is used to generate the polymer. In some embodiments, three or more starting materials (e.g., three or more different chemical entities) are used to generate the polymer. In some embodiments, approximately the same amounts of the three or more starting materials are used to generate the polymer. For example, the first, second, and third starting material are combined in approximately equal amounts to provide the polymer. In other embodiments, a greater amount of the first material is used to generate the polymer, and/or a greater amount of the first and second materials are used to generate the polymer.

In some embodiments, starting materials are mixed (e.g., via centrifugation, agitation, stirring, or other mechanical or manual mixing) upon combination to provide the polymer (e.g., biodegradable polymer).

In some embodiments, the polymer (e.g., biodegradable polymer)forms immediately upon combining the starting materials. In some embodiments, combining the starting materials provides a liquid solution, which liquid solution gels over a period of time (e.g., over several minutes or hours, and/or upon application to a treatment area). In some embodiments, combining the starting materials provides a liquid solution, which liquid solution gels after introduction of a gelling agent.

In some embodiments, one or more starting materials are provided in a solution. For example, one or more starting materials are provided in a buffered solution. In some embodiments, a buffered solution is a hydrogen phosphate buffer solution (e.g., a sodium hydrogen phosphate buffer solution). In some embodiments, one or more starting materials is provided in ethanol and/or water, such as a 50/50 water/ethanol solution.

In some embodiments, a polymerizing aid is combined with the starting materials to facilitate generation of the polymer (e.g., biodegradable polymer). In some cases, the polymerizing aid is acetonitrile.

In some embodiments, the polymer is generated under standard temperature and pressure conditions (e.g., ambient temperature and pressure). In some embodiments, the polymer is generated at an elevated temperature (e.g., greater than 25 degrees Celsius, ° C.). In some embodiments, the polymer is generated at a pH between about 6 to about 9, such as between about 6 to about 7, between about 7 to about 8, or between about 8 to about 9. In some embodiments, the polymer is generated at a pH between about 7.5 to about 9, such as between about 7.5 to about 8, between about 8 to about 8.5, between about 8 to about 9, or between about 8.5 to about 9.

In some embodiments, the starting materials comprise one or more materials that are not polymerizable. For example, the starting materials include an agent such as a therapeutic agent (e.g., steroid, immunotherapeutic agent, decongestant, coagulating agent, or other agent) or a patency maintaining agent. Details of such agents are provided in a subsequent section. In some embodiments, an agent (e.g., a therapeutic agent) is combined with the starting materials prior to formation of the polymer. In other embodiments, an agent (e.g., a therapeutic agent) is added after formation of the polymer.

In some embodiments, a polymer (e.g., biodegradable polymer) is or comprises a Michael addition polymer, i.e., a polymer formed using a Michael reaction. The Michael donor is a hetero-atomic nucleophile, selected from the group consisting of nitrogen, sulfur, oxygen or phosphorous nucleophiles.

In some embodiments, the Michael donor is a thiol donor and the reaction is a thiol-Michael addition reaction. In some embodiments, the thiol donor is an aromatic thiol. In some embodiments, the aromatic thiol has more than one aromatic ring, i.e., n>1.

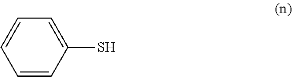

In some embodiments, the Michael donor is a multi-arm donor, such as a compound including multiple aromatic thiol moieties, such as, for example:

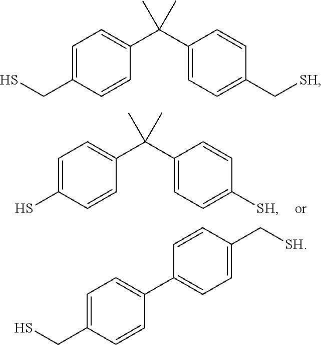

In some embodiments, the Michael donor does not include an aromatic thiol moiety but instead includes one or more non-aromatic thiol moieties. In some embodiments, the Michael donor is a compound including three or more non-aromatic thiol moieties. In some embodiments, the Michael donor is a compound including four or more non-aromatic thiol moieties, such as six non-aromatic thiol moieties or eight non-aromatic thiol moieties. In some embodiments, the Michael donor comprises three or more of the following moieties:

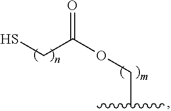

wherein n and m are each independently selected from the group consisting of 1, 2, and 3. In some embodiments, the Michael donor comprises four or more such moieties, such as six such moieties or eight such moieties. In some embodiments, the Michael donor comprises three or more of the following moieties:

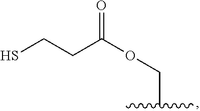

wherein n and m are each independently selected from the group consisting of 1, 2, and 3. In some embodiments, the Michael donor comprises four or more such moieties, such as six such moieties or eight such moieties. In some embodiments, the Michael donor is represented by the formula:

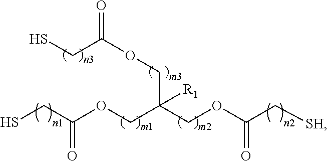

wherein R₁ is selected from the group consisting of H and C₁-C₆ alkyl; n₁, n₂, and n₃ are each independently selected from the group consisting of 1, 2, and 3; and m₁, m₂, and m₃ are each independently selected from the group consisting of 1, 2, and 3. In some embodiments, R₁ is C₁ alkyl. In some embodiments, n₁, n₂, and n₃ are each 2. In some embodiments, m₁, m₂, and m₃ are each 1. In some embodiments, the Michael donor is a compound represented by the formula:

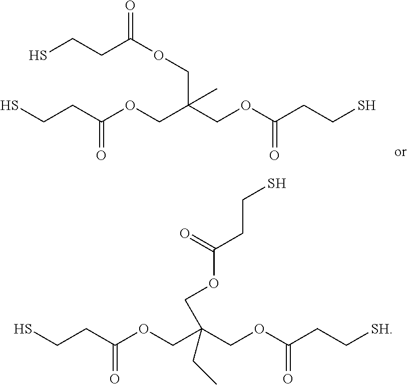

In other embodiments, the Michael donor comprises three or more of the following moieties:

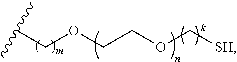

wherein k and m are each independently selected from the group consisting of 1, 2, and 3 and wherein n is selected from the group consisting of 1-500. In some embodiments, the Michael donor comprises four or more such moieties, such as six such moieties or eight such moieties. In some embodiments, the Michael donor comprises three or more of the following moieties:

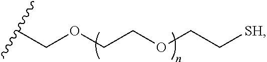

wherein n is selected from the group consisting of 1-500. In some embodiments, the Michael donor comprises four or more such moieties, such as six such moieties or eight such moieties. In some embodiments, the Michael donor is a compound represented by the formula:

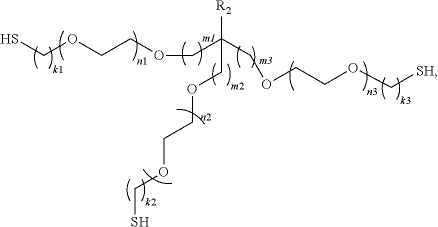

where R₂ is selected from H, C₁₋₆ alkyl, and

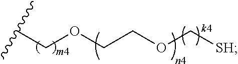

n₁, n₂, n₃, and n₄ (if present) are each independently selected from the group consisting of 1-500, such as 1-20; m₁, m₂, m₃, and m₄ (if present) are each independently selected from the group consisting of 1, 2, and 3; and k₁, k₂, k₃, and k₄ (if present) are each independently selected from the group consisting of 1, 2, and 3. b In some embodiments, the Michael donor is a compound represented by the formula:

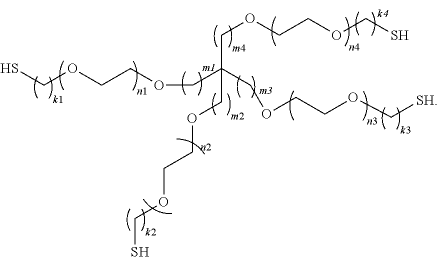

In some embodiments, m₁, m₂, m₃, and m₄ are each 1. In some embodiments, k₁, k₂, k₃, and k₄ are each 2. In some embodiments, the Michael Donor is a compound represented by the formula:

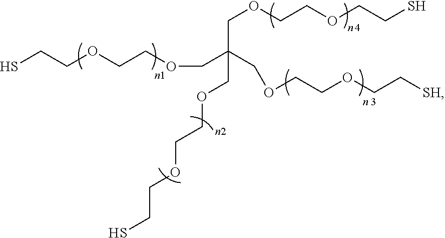

where n₁, n₂, n₃, and n₄ are each independently selected from the group consisting of 1-500. In some embodiments, two or more of n₁, n₂, n₃, and n₄ are the same. In some embodiments, n₁, n₂, n₃, and n₄ are all the same.

In some embodiments, the Michael donor does not include a thiol moiety. In some embodiments, a Michael donor includes one or more oxirane moieties. In some embodiments, the Michael donor is a compound represented by the formula:

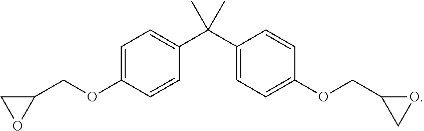 [SMILES: CC(C)(c1ccc(OCC2CO2)cc1)c1ccc(OCC2CO2)cc1]

In some embodiments, the Michael acceptor is an electron-deficient ene selected from the group consisting of acrylates, methacrylates, vinyl sulfones, and maleimides. In some embodiments, the Michael acceptor comprises one or more polyethylene glycol moieties.

In some embodiments, the Michael acceptor is a multi-arm acrylate. In some embodiments, the multi-arm acrylate has greater than 2, greater than 3, greater than four, greater than five, greater than six, or greater than seven arms. In some embodiments, the Michael acceptor includes six acrylate arms. In some embodiments, the Michael acceptor includes eight acrylate arms. In some embodiments, an arm of a Michael acceptor is represented by the formula:

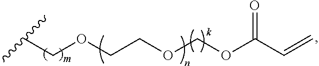

wherein n is selected from the group consisting of 1-500, such as 1-20; and m and k are each independently selected from the group consisting of 1, 2, and 3. In some embodiments, a Michael acceptor includes four such moieties. In some embodiments, a Michael acceptor includes six such moieties. In some embodiments, a Michael acceptor includes eight such moieties. In some embodiments, an arm of a Michael acceptor is represented by the formula:

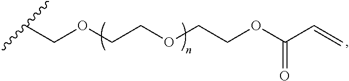

wherein n is selected from the group consisting of 1-500, such as 1-20. In some embodiments, a Michael acceptor includes four such moieties. In some embodiments, a Michael acceptor includes six such moieties. In some embodiments, a Michael acceptor includes eight such moieties. In some embodiments, the Michael acceptor is a compound represented by the formula:

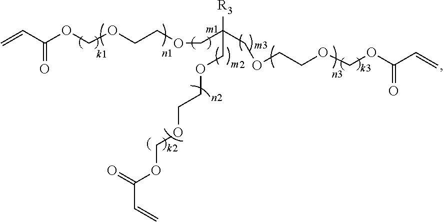

wherein R₃ is selected from the group consisting of H, C₁-C₆ alkyl, and

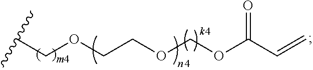

n₁, n₂, n₃, and n₄ (if present) are each independently selected from the group consisting of 1-500, such as 1-20; m₁, m₂, m₃, and m₄ (if present) are each independently selected from the group consisting of 1, 2, and 3; and k₁, k₂, k₃, and k₄ (if present) are each independently selected from the group consisting of 1, 2, and 3. In some embodiments, the Michael acceptor is a compound represented by the formula:

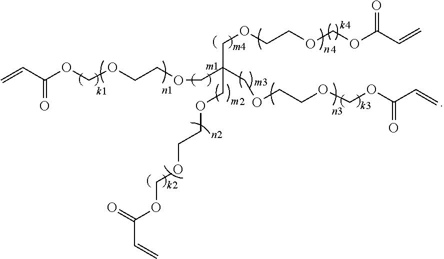

In some embodiments, m₁, m₂, m₃, and m₄ are each 1. In some embodiments, k₁, k₂, k₃, and k₄ are each 2. In some embodiments, the Michael acceptor is a compound represented by the formula:

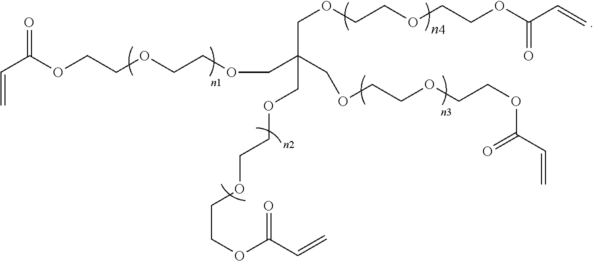

In some embodiments, an arm of a Michael acceptor is represented by the formula

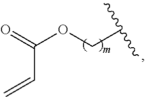

wherein m is selected from the group consisting of 1, 2, and 3. In some embodiments, a Michael acceptor includes four such moieties. In some embodiments, a Michael acceptor includes six such moieties. In some embodiments, a Michael acceptor includes eight such moieties. In some embodiments, the Michael acceptor is a compound represented by the formula:

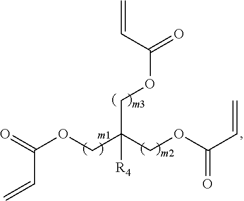

wherein R₄ is selected from the group consisting of H, C₁-C₆ alkyl, and C₁-C₆ hydroxyalkyl; and m₁, m₂, and m₃ and are each independently selected from the group consisting of 1, 2, and 3. In some embodiments, R₄ is selected from C₁-C₆ hydroxyalkyl. In some embodiments, R₄ is C₁ hydroxyalkyl. In some embodiments, m₁, m₂, and m₃ and are each 1. In some embodiments, the Michael acceptor is a compound represented by the formula:

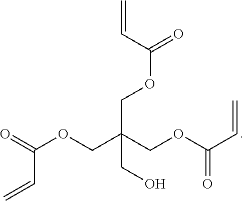 [SMILES: C=CC(=O)OCC(CO)(COC(=O)C=C)COC(=O)C=C]

In some embodiments, the Michael acceptor is a compound represented by the formula:

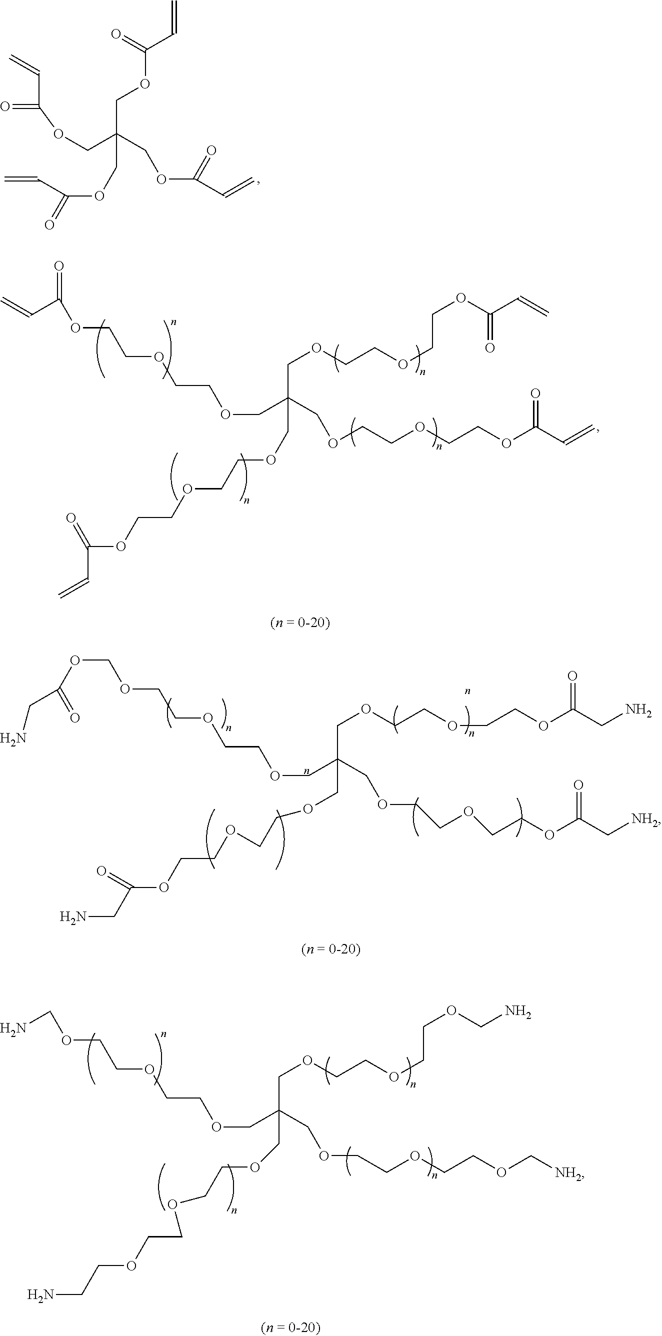

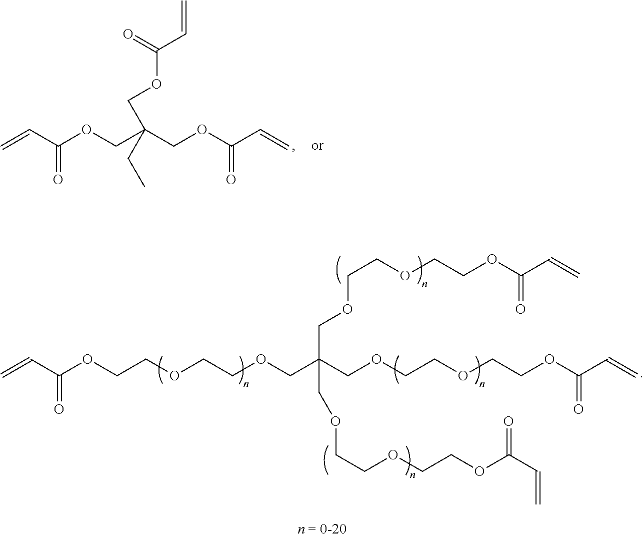

Any useful combination of Michael donors and Michael acceptors can be used in a process of preparing a polymer (e.g., biodegradable polymer) of the present disclosure. In some embodiments, a single Michael donor and a single Michael acceptor are used to prepare the polymer. In some embodiments, a single Michael donor and two or more Michael acceptors are used to prepare the polymer. In some embodiments, two or more Michael donors and a single Michael acceptor are used to prepare the polymer.

In some embodiments, the Michael donor is represented by the formula:

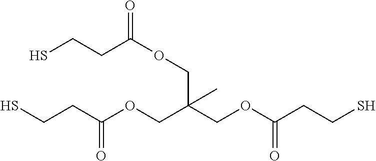 [SMILES: CC(COC(=O)CCS)(COC(=O)CCS)COC(=O)CCS]

and the Michael acceptor is represented by the formula:

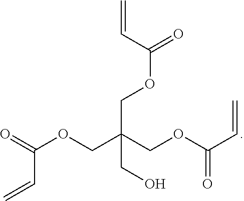 [SMILES: C=CC(=O)OCC(CO)(COC(=O)C=C)COC(=O)C=C]

In some embodiments, the Michael donor is represented by the formula:

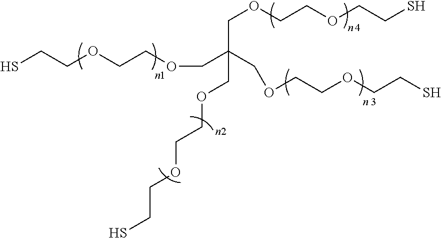

and the Michael acceptor is represented by the formula:

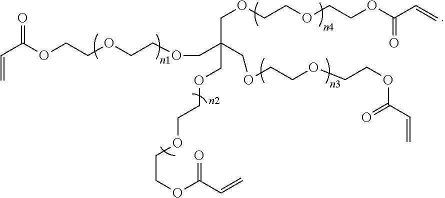

In some embodiments, the Michael donor(s) is represented by the formula:

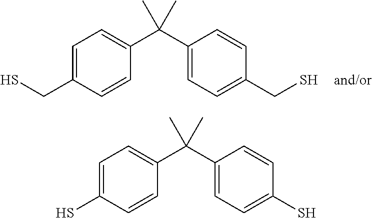

and the Michael acceptor(s) is represented by the formula:

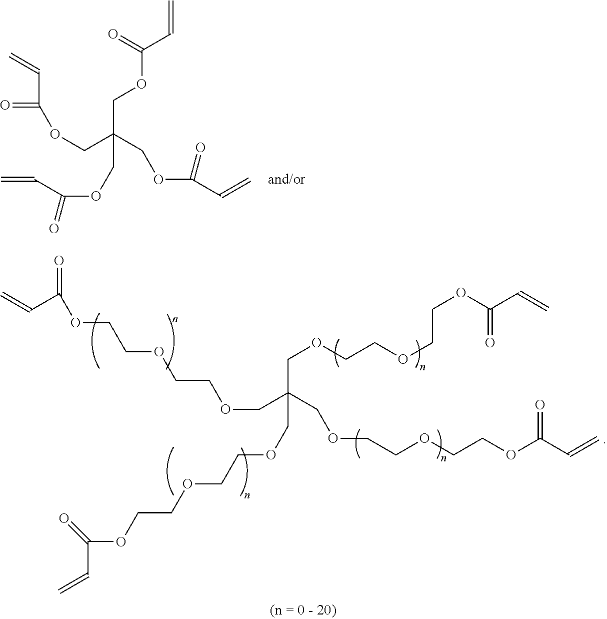

In some embodiments, the Michael donor(s) is represented by the formula:

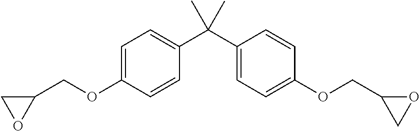 [SMILES: CC(C)(c1ccc(OCC2CO2)cc1)c1ccc(OCC2CO2)cc1]

and the Michael acceptor(s) is represented by the formula:

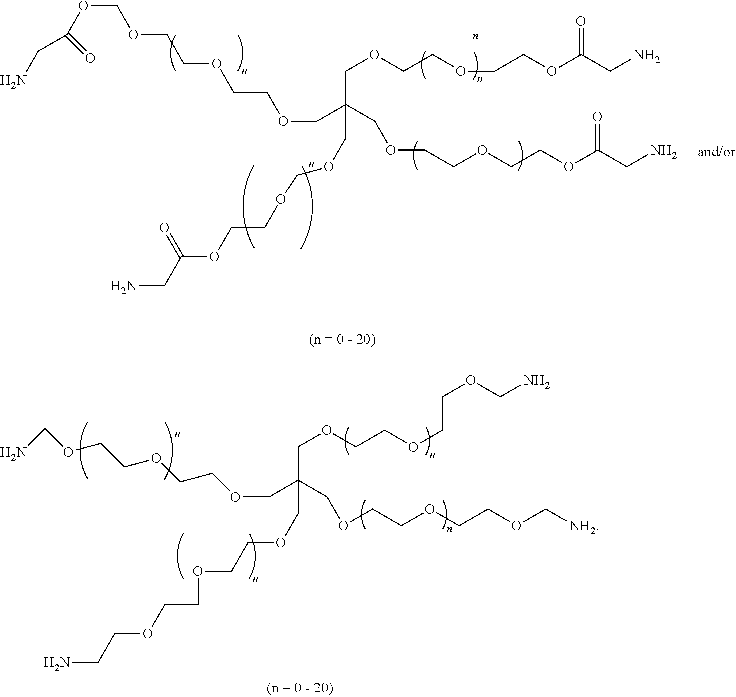

In some embodiments, the Michael donor is represented by the formula:

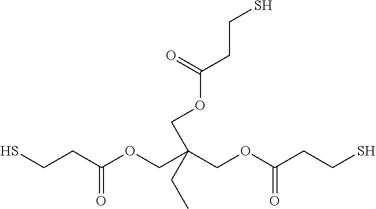 [SMILES: CCC(COC(=O)CCS)(COC(=O)CCS)COC(=O)CCS]

and the Michael acceptors are represented by the formulas:

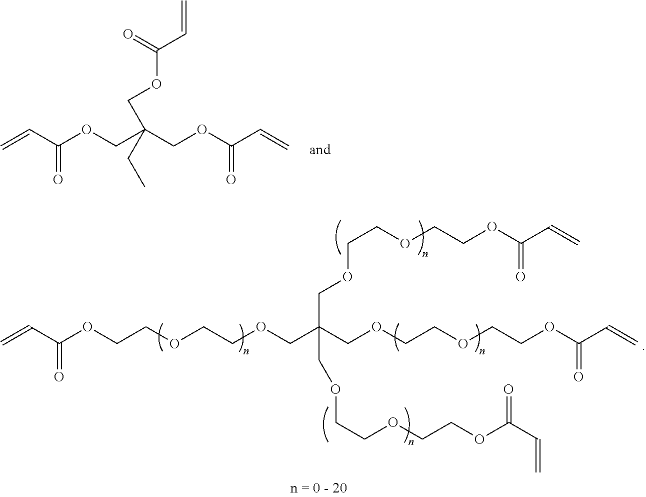

The thiol-Michael addition reaction progresses via an anionic pathway. In some embodiments, the reaction uses of a catalyst. Examples of catalysts useful in the reaction include a base, a nucleophile, or a Lewis acid. In alternative embodiments, the reaction is performed without a catalyst using highly polar solvent. The rate limiting step in this reaction is generally the nucleophilic action of the thiolate anion to a vinyl.

In some embodiments, the reaction is base catalyzed (e.g., an amine) to facilitate the reaction between a thiol and an electron-deficient vinyl group to yield a thiolether additional product. The reaction kinetics are dependent on one or more of the following: strength and concentration of the catalyst, the thiol pKa, the steric accessibility of the thiol, nature of the electron withdrawing group coupled to the C═C bond and the polarity and pH of the solvent.

In some embodiments, the polymer is prepared by the reaction of bis-dithiol and a multi-armed acrylate in a Michael reaction. The aromatic ring provides hardness to the material.

In some embodiments, the polymer is prepared by the reaction shown in Scheme 1 below:

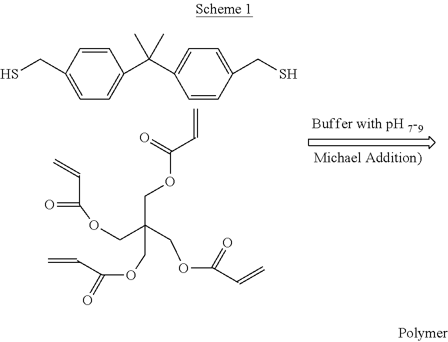

In some embodiments, the polymer is prepared by the reaction shown in Scheme 2 below:

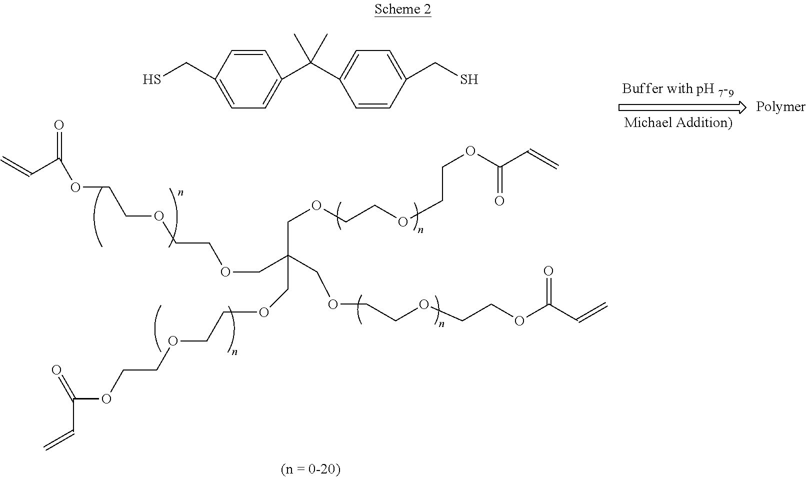

Depending on the modulus of the materials/device desired, the value of n for the PEG unit is adjusted to meet the requirements. For example, if the modulus of the material is desired to be more than 20 megapascal (MPa), such materials are prepared by having PEG chains length of about 5 units, i.e., n=5. If however, however, modulus of less than 1 MPa, then n=10-20. For modulus of 20-1000 MPA, the value of n=0-5. In some embodiments, each n is the same. In other embodiments, one or more n's are different from one another.

In other embodiments, the polymer is prepared by the reaction shown in Scheme 3 below:

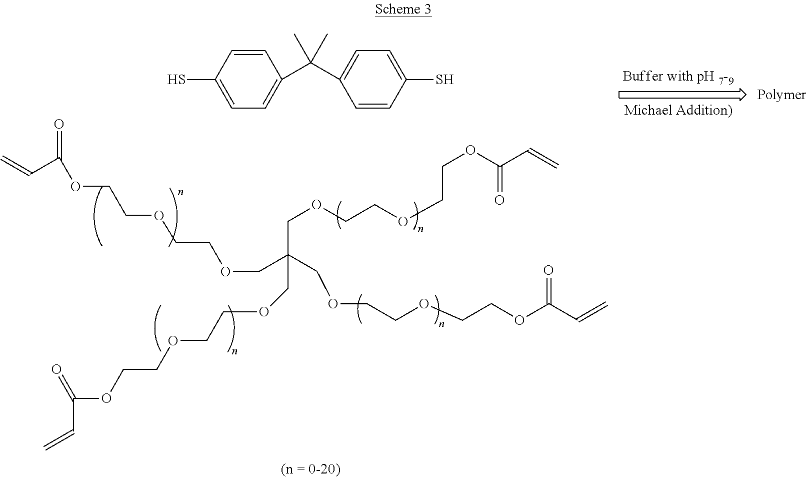

In some embodiments, each n is the same. In other embodiments, one or more n's are different from one another.

In some embodiments, the polymer is prepared by the reaction of bis-gycildyl ether and an amine.

In another embodiment, the polymer is prepared by the reaction shown in Scheme 4 below:

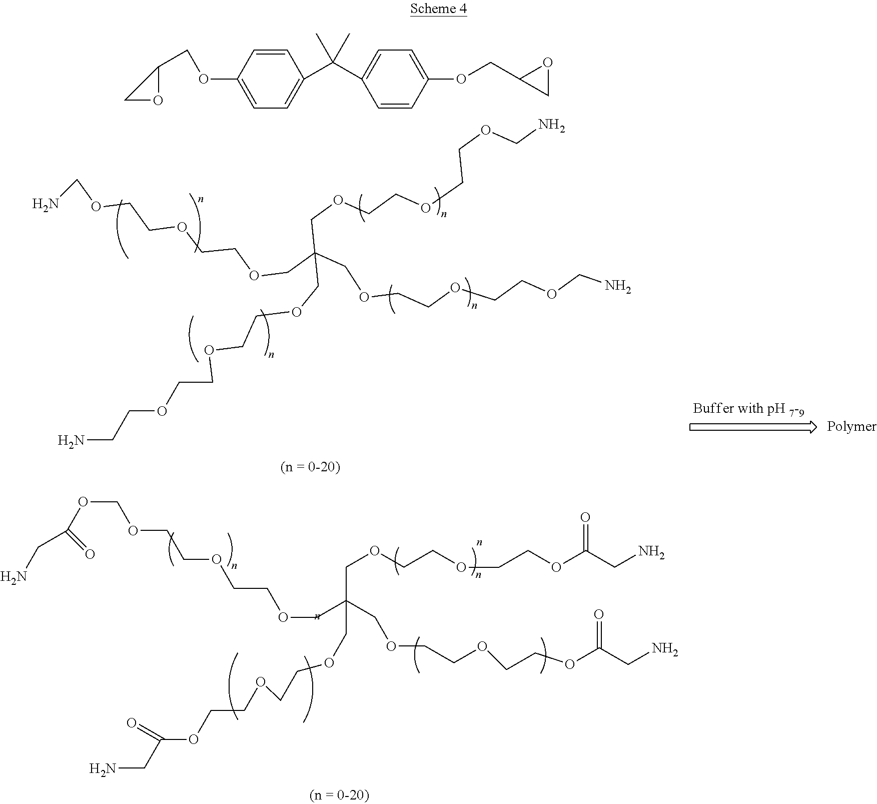

In some embodiments, both an amine containing two ester linkages and a non-ester amine are utilized. By increasing the non-ester containing amine, the degradation time is increased whereas by increasing the ester containing amine, the degradation is decreased. In some embodiments, the polymer (e.g., biodegradable polymer) degrades in between about 2 and about 8 weeks, or more particularly, about 4 and about 8 weeks.

In some embodiments, each n of a given compound used in the reaction is the same. In other embodiments, one or more n's of a given compound used in the reaction are different from one another.

In some embodiments, the polymer is prepared by the reaction of a thiol-containing compound, a compound comprising one or more acrylate moieties, and a compound comprising one or more acrylate moieties and one or more polyethylene glycol moieties, as shown in Scheme 5 below:

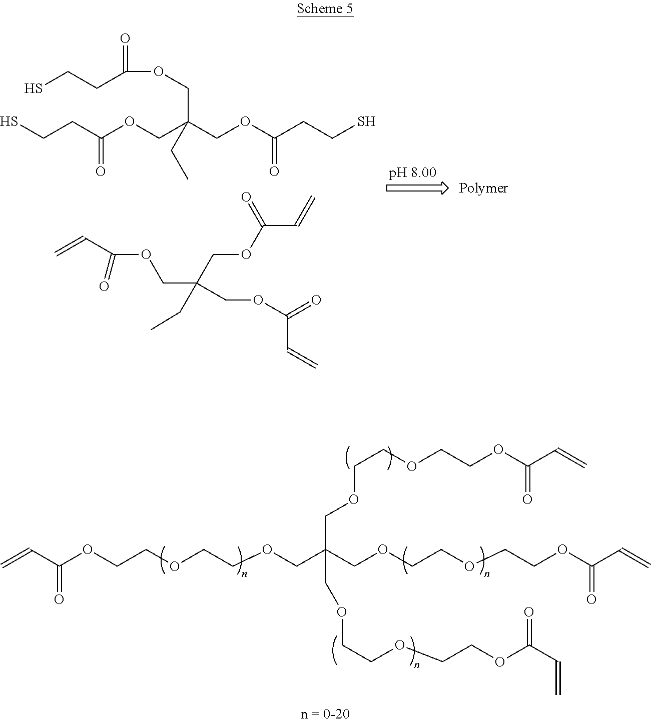

In some embodiments, each n=1-500. In some embodiments, each n=1-20, such as between 1 and 20. In some embodiments, each n is the same. In other embodiments, one or more n's are different from one another.

In some embodiments, the polymer is prepared by the reaction shown in Scheme 6 below:

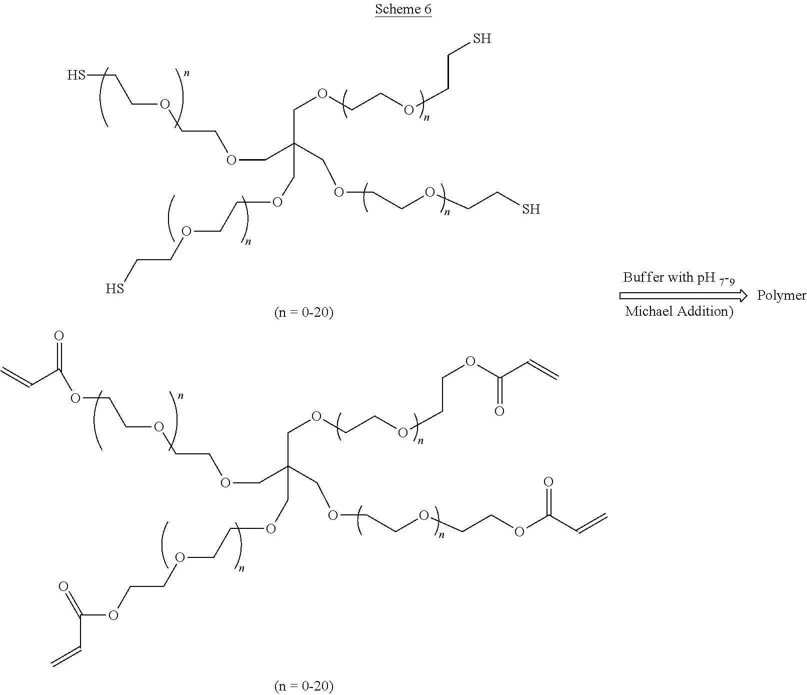

In some embodiments, each n=1-500. In some embodiments, each n=1-20, such as between 1 and 20. In some embodiments, each n is the same. In other embodiments, one or more n's are different from one another.

In some embodiments, the polymer is prepared by the reaction shown in Scheme 7 below:

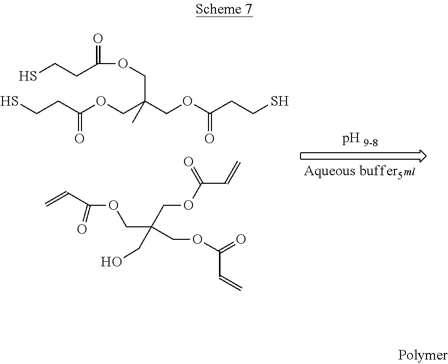

In some embodiments, a polymer (e.g., biodegradable polymer) provided herein is absorbed by the body over 2-8 weeks, such as over 2-4 weeks.

In some embodiments, a polymer (e.g., biodegradable polymer) provided herein includes an agent such as a therapeutic agent. In some embodiments, the agent is included a gel matrix comprising the polymer.

In some embodiments, a polymer (e.g., biodegradable polymer) provided herein is lyophilized or freeze dried. In some embodiments, the polymer is lyophilized or freeze dried in the presence of a cryoprotectant such as dimethyl sulfoxide, ethylene glycol, glycerol, 2-methyl-2,4-pentanediol, propylene glycol, sucrose, or trehaolse. In some embodiments, a lyophilized or freeze dried polymer is rehydrated prior to administration or application of the polymer to a treatment site.

### Compositions and Devices

In an aspect, the present disclosure provides a composition comprising a polymer (e.g., a biodegradable polymer) as described herein. In some embodiments, a composition comprises a single polymer (e.g., biodegradable polymer) type. In some embodiments, a composition comprises two or more polymer types, such as two or more polymer types that can interact with one another to form, e.g., a matrix.

In some embodiments, a composition comprises one or more materials in addition to the polymer (e.g., biodegradable polymer). In some embodiments, a composition comprises a solvent such as water and/or ethanol. In some embodiments, a composition comprises a buffered solution such as a sodium hydrogen phosphate buffered solution.

In another aspect, the present disclosure provides a material or device comprising a polymer (e.g., a biodegradable polymer) as described herein. In some embodiments, the material or device comprises an absorbent material. In some embodiments, the material of the present disclosure comprises a gelled polymer (e.g., biodegradable polymer). In some embodiments, the device of the present disclosure comprises a gelled polymer and an applicator (e.g., tab, syringe, container, or other applicator), removable packaging, or other component. In some embodiments, the device is an absorbent pad. An absorbent pad can have any useful size and shape and can be tailored for a particular application. In some embodiments, the size of a device is selected based on the intended patient population (e.g., child vs. adult) and/or the intended treatment area (e.g., nasal passage or other bodily area). For example, an absorbent pad for application to a wound in a nasal passage can be a different size than an absorbent pad for application to a wound in another area of the body. The device of the present disclosure is generally only a few millimeters to a few centimeters in diameter. In some embodiments, the device (e.g., absorbent pad) is about 1 mm, about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, about 40 mm, about 50 mm, or more in diameter.

In some embodiments, a composition, material, or device comprises an agent such as a therapeutic agent. In some embodiments, a therapeutic agent is selected from the group consisting of a corticosteroid, a decongestant, an immunotherapeutic agent, an adrenocorticoid (e.g., corticosteroid or steroid), an analgesic agent, an analgesic adjunct, an analgesic-anesthetic, an anesthetic, an antibiotic, an antibacterial agent, an anti-infective agent, an antibiotic therapy adjunct, an antidote, an anti-emetic agent, an anti-fungal agent, an anti-inflammatory agent, an anti-vertigo agent, an anti-viral agent, a biological response modifier, a cytotoxic agent, a diagnostic aid, an immunizing agent, an immunomodulator, proteins, and peptides. In some embodiments, a composition, material, or device comprises a therapeutic agent useful in the treatment of a condition such as a nasal condition. In some embodiments, a composition, material, or device comprises a therapeutic agent useful in promoting hemostasis, such as a blood clotting agent or coagulant. In some embodiments, a composition, material, or device comprises an agent selected from the group consisting of von Wiillebrand factor, platelet activating factors, fibrin, fibrinogen, a blood coagulation factor (e.g., FVII (stable factor), FIX (Christmas factor), FX, FXI (plasma thromboplastin), FXII (Hageman factor), FXIII (fibrin stabilizing factor), FVIII, and subtypes thereof), tissue factor, kallikrein, prekallikrein, thrombin, desmopressin, prothrombin complex concentrate, recombinant activated human factor VII, and other agents useful in promoting blood coagulation. In some embodiments, a composition , material, or device comprises an adsorbent chemical such as a zeolite, a hemostatic agent (e.g., antifibrinolytic agent, vitamin K, microfibrillar collagen, chitosan, anhydrous aluminum sulfate, and kaolin), and/or another procoagulant such as tranexamic acid, aminocaproic acid, or aprotinin.

Non-limiting examples of adrenocorticoids for use in compositions, devices, and materials of the present disclosure include betamethasone, cortisone, dexamethasone, hydrocortisone, methylprednisolone, paramethasone, prednisolone, prednisone, and triamcinolone. Non-limiting examples of analgesics for delivery by the device (e.g., pressure equalization tube) of the present disclosure include acetaminophen, aspirin, buprenorphine, butalbital, butorphanol, codeine, dezocine, diflunisal, dihydrocodeine, etodolac, fenoprefen, fentanyl, floctafenine, hydrocodone, hydromorphone, ibuprofen, ketoprofen, ketorolac, levorphanol, magnesium salicylate, meclofenamate, mefenamic acid, meperidine, meprobamate, methadone, methotrimeprazine, morphine, nalbuphine, naproxen, opium, oxycodone, oxymorphone, pentazocine, phenobarbital, propoxyphene, salsalate, and sodium salicylate.

Non-limiting examples of analgesics for use in compositions, devices, and materials of the present disclosure include antipyrine and benzocaine.

Non-limiting examples of anesthetics for use in compositions, devices, and materials of the present disclosure include bupivicaine, tetracaine, procaine, proparacaine, propoxycaine, dimethocaine, cyclomethycaine, chloroprocaine, benzocaine, lidocaine, prilocaln, levobupivicaine, ropivacaine, dibucaine, articaine, carticaine, etidocaine, mepivacaine, piperocaine, and trimecaine.

Non-limiting examples of antibiotics for use in compositions, devices, and materials of the present disclosure include ciprofloxacin, cefuroxime, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, enoxacin, gatifloxacin, levofloxacin, lomefloxacin, moxifloxacin, norfloxacin, ofloxacin, trovafloxacin, bacitracin, colistin, polymyxin B, azithromycin, clarithromycin, dirithromycin, erythromycin, roxithromycin, troleandomycin, telithromycin, spectinomycin, amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mafenide, sulfacetamide, sulfamethizole, sulfasalazine, sulfisoxazole, trimethoprim, and trimethoprim-sulfamethoxazole. In some embodiments, the antibiotic is ciprofloxacin.

Non-limiting examples of anti-fungal drugs for use in compositions, devices, and materials of the present disclosure include amphotericin B, caspofungin, clotrimazole, fluconazole, itraconazole, ketoconazole, voriconazole, terbinafine, and nystatin.

Anti-inflammatory agents for use in compositions, devices, and materials of the present disclosure include both non-steroidal and steroidal anti-inflammatory agents. Representative, non-limiting examples of non-steroidal anti-inflammatory agents include acetic acids, COX-2 inhibitors, fenamates, oxicam derivatives, propionic acids and salicylates. In some embodiments, the acetic acid is selected from the group consisting of diclofenac, etodolac, indomethacin, ketorolac, nabumetone, sulindac, and tolmetin. In some embodiments, the COX-2 inhibitor is celecoxib. In some embodiments, the fenamate is selected from the group consisting of meclofenamate and mefanamic acid. In some embodiments, the oxicam derivative is selected from the group consisting of meloxicam and piroxicam. In some embodiments, the propionic acid is selected from the group consisting of fenoprofen, flurbiprofen, ibuprofen, ketoprofen, naproxen and oxaprozin. In some embodiments, the salicylate is selected from the group consisting of aspirin, diflunisal, magnesium salicylate and salsalate. Representative, non-limiting examples of steroidal active agents dexamethasone, dexamethasone sodium phosphate, hydrocortisone, hydrocortisone acetate, prednisolone, methylprednisolone, prednisone, triamcinolone acetonide, mometasone, budesonide, fluocinolone acetonide, betamethasone, betamethasone sodium phosphate, betamethasone valerate, cortisone acetate, isoflupredone acetate, tixocortol pivalate, triamcinolone alcohol, amcinonide, desonide, fluocinonide, halcinonide, fluocortolone, hydrocortisone-17-butyrate, hydrocortisone-17-valerate, aclometasone dipropionate, betamethasone dipropionate, prednicarbate, clobetasone-17-butyrate, clobetasol-17-propionate, fluocortolone caproate, fluocortolone pivalate, and fluprednidene.

In some embodiments, a composition, device, or material of the present disclosure is configured to deliver at least one antibiotic. In some embodiments, the antibiotic is a penicillin. In some embodiments, the penicillin is amoxicillin. In some embodiments, the antibiotic is a macrolide. In some embodiments, the macrolide is selected from the group consisting of azithromycin, clarithromycin, erythromycin, fidaxomicin or telithromycin. In some embodiments, the antibiotic is a ketolide or a flouroketolide.

In some embodiments, a composition, device, or material of the present disclosure is configured to deliver at least one antibiotic, where the at least one antibiotic is a cephalosporin. In some embodiments, the cephalosporin is a second generation cephalosporin, a third generation cephalosporin, a fourth generation cephalosporin, or a fifth generation cephalosporin. In some embodiments, the second generation cephalosporin is selected from the group consisting of cefaclor, cefonicid, cefprozil, cefuroxime, cefuzonam, cefmetazole, cefotetan, cefoxitin, carbacephems (e.g., loracarbef), and cephamycins (e.g., cefbuperazone). In some embodiments, the third generation cephalosporin is selected from the group consisting of cefcapene, cefdaloxime, cefdinir, cefditoren, cefetamet, cefixime, cefmenoxime, cefodizime, cefotaxime, cefovecin, cefpimizole, cepodoxime, cefteram, ceftamere, ceftibuten, ceftiofur, ceftiolene, ceftizoxime, ceftriazone, cefoperazone, ceftazidime, and oxacephems (e.g., latamoxef). In some embodiments, the fourth generation is selected from the group consisting of cefclidine, cefepime, cefluprenam, cefoselis, cefozopran, cepirome, cefquinome, and oxacephems (e.g., flomoxef). In some embodiments, the fifth generation cephalosporin is selected from the group consisting of ceftobiprole, ceftaroline, and ceftolozane. In some embodiments, the cephalosporin is selected from the group consisting of cefaloram, cefaparole, cefcanel, cefedrolor, cefempidone, cefetrizole, cefivitril, cefmatilen, cemedpidium, cefoxazole, cefrotil, cefsumide, ceftioxide, cefuracetime, and nitrocefin.

In some embodiments, a composition, device, or material of the present disclosure is configured to deliver at least two antibiotics. In some embodiments, the at least two antibiotics are a penicillin (e.g., amoxicillin) and a cephalosporin (e.g., a second, third, fourth, or fifth generation cephalosporin). In some embodiments, the at least two antibiotics comprise sulfa-based combinations, e.g., erythromycin-sulfisoxazole or trimethoprim-sulfamethoxazole.

In some embodiments, a composition, device, or material of the present disclosure is configured to deliver at least two antibiotic agents and at least one steroid. In some embodiments, the steroid is selected from the group consisting of momentasone furoate and prednisolone.

In some embodiments, a composition, device, or material of the present disclosure is configured to deliver at least one anti-viral agent and at least one steroid.

In some embodiments, a device of the present disclosure comprises a tube (e.g., pressure equalization tube), an ear plug, a nose plug, a hearing aid, an amplifier, or an implantable device. In some embodiments, the device is suitable for administration to and/or placement in a nasal passage or ear. For example, in some embodiments, the device is a tube (e.g., pressure equalization tube) made from one or more biodegradable polymers of the present disclosure. In other embodiments, the device is a tube (e.g., a pressure equalization tube) coated with one or more biodegradable polymers. In some embodiments, the device (e.g., pressure equalization tube) may comprise a therapeutic agent and/or a patency maintaining agent. In some embodiments, a device (e.g., tube) does not need to be removed surgically and, in certain embodiments, is drug-eluting. For example, in some embodiments, the device elutes at least one agent (e.g., a patency maintaining agent) capable of maintaining the patency of a passage or incision for an extended period of time. Alternatively or in addition, the device elutes one or more therapeutic agents (e.g., as described herein).

The material(s) used to form and/or coat the device (e.g., tube, such as a pressure equalization tube) includes one or more biodegradable polymers. The material has a hardness to indentation suitable for withstanding insertion or implantation forces without buckling or bending and for maintaining the passage in a substantially unobstructed, open condition to permit drainage and ventilation of a passage (e.g., nasal or ear passage) over extended periods of time.

Hardness of a material (e.g., a polymer provided herein or a combination thereof) can be measured by, for example, durometer. Higher numbers indicate harder materials; lower numbers indicate softer materials. There are several scales of durometer used for materials with different properties. The two most common scales are the ASTM D2240 type A and type D scales. The A scale is for softer plastics, while the D scale is for harder ones. Each scale results in a value between 0 and 100, with higher values indicating a harder material.

In some embodiments, a material (e.g., a material comprising one or more biodegradable polymers) has a durometer (hardness) of greater than about 40, greater than about 50, greater than about 60, greater than about 70, greater than about 80, greater than about 85, greater than about 90, greater than about 95, or about 100. In some embodiments, a material has a hardness of between about 80 and about 100, more particularly about 85 and 100, and even more particularly, about 90 and 100. In some embodiments, a device (e.g., pressure equalization tube) formed of one or more biodegradable polymers has a hardness of between about 40 and about 100, more particularly about 45 and about 95, or more particularly, about 50 and about 90.

In some embodiments, the material (e.g., biodegradable polymer(s)) is compatible with one or more therapeutic agents (e.g., as described herein). For example, a biodegradable polymer does not and should not destabilize the therapeutic agent or affect its performance.

In some embodiments, the material (e.g., biodegradable polymer(s)) is compatible with an agent for maintaining patency of a passage or aperture (e.g., as described herein).

### Methods of Treating Nasal Conditions and Promoting Hemostasis

In some embodiments, the polymers (e.g., biodegradable polymers) of the present disclosure and materials and devices comprising the same are useful for treating a condition of the ear, nose, and/or throat, such as a nasal condition. In some embodiments, a condition of the ear, nose, and/or throat is selected from the group consisting of achalasia, acoustic neuroma/removal of acoustic neuroma, enlarged adenoids/adenoidectomy, infection, cancer, Meniere's disease, hay fever, allergies, asthma, sinusitis, diphtheria, and nasal polyps. In some embodiments, a nasal condition is selected from the group consisting of sinusitis, nasal polyps, nasal obstruction, rhinitis, bacterial infection, nosebleed, and nasal tumors. In some embodiments, a nasal condition is sinusitis, such as acute sinusitis, recurrent sinusitis, and chronic sinusitis. In some embodiments, an individual treated with a composition or device of the present disclosure has undergone a surgical procedure, such as a surgical procedure to remove one or more tumors (e.g., nasal tumors), inflamed glands (e.g., adenoid, thyroid, or tonsil), or nasal polyps. In such an instance, the composition or device is used to promote hemostasis in the individual.

In some embodiments, a subject treated with a composition or device of the present disclosure has one or more symptoms selected from the group consisting of bleeding (e.g., uncontrolled bleeding), anosmia, dysosmia, ageusia, hypogeusia, dysgeusia, nasal congestion, nasal obstruction, balance problems, auditory trauma, hearing loss, cough, dizziness, vertigo, allergies, asthma, rash, sore throat, tinnitus, nasal polyps, nosebleed, and tumors (e.g., nasal tumors).

In some embodiments, a composition or device for treating a condition (e.g., a nasal condition) comprises an agent useful in the treatment of the condition (e.g., as described herein). In some embodiments, the agent is selected from a patency maintaining agent and a therapeutic agent (e.g., as described herein). In some embodiments, a therapeutic agent is an agent useful in promoting hemostasis and/or coagulation (e.g., as described herein). In some embodiments, the therapeutic agent is selected from the group consisting of a corticosteroid, a decongestant, an immunotherapeutic agent, an adrenocorticoid (e.g., corticosteroid or steroid), an analgesic agent, an analgesic adjunct, an analgesic-anesthetic, an anesthetic, an antibiotic, an antibacterial agent, an anti-infective agent, an antibiotic therapy adjunct, an antidote, an anti-emetic agent, an anti-fungal agent, an anti-inflammatory agent, an anti-vertigo agent, an anti-viral agent, a biological response modifier, a cytotoxic agent, a diagnostic aid, an immunizing agent, an immunomodulator, proteins, and peptides.

In some embodiments, a composition or device for promoting hemostasis comprises an agent useful in promoting hemostasis. In some embodiments, the agent is selected from a patency maintaining agent and a therapeutic agent (e.g., as described herein). In some embodiments, a therapeutic agent is an agent useful in promoting hemostasis and/or coagulation (e.g., as described herein). In some embodiments, the agent is selected from the group consisting of von Willebrand factor, platelet activating factors, fibrin, fibrinogen, a blood coagulation factor (e.g., FVII (stable factor), FIX (Christmas factor), FX, FXI (plasma thromboplastin), FXII (Hageman factor), FXIII (fibrin stabilizing factor), FVIII, and subtypes thereof), tissue factor, kallikrein, prekallikrein, thrombin, desmopressin, prothrombin complex concentrate, recombinant activated human factor VII, and other agents useful in promoting blood coagulation, such as a zeolite or a hemostatic agent.

In some embodiments, a device of the present disclosure comprises a polymer (e.g., biodegradable polymer) of the present disclosure. In some embodiments, a device of the present disclosure comprises a composition comprising a polymer (e.g., biodegradable polymer) of the present disclosure. In some embodiments, the polymer or the composition comprising the polymer is integrated throughout the device. In some embodiments, the polymer or the composition comprising the polymer is localized in one or more regions of the device. In an example, the device is an absorbent pad and the polymer or the composition comprising the polymer is distributed along a surface of the absorbent pad that is configured to contact a wound area. In some embodiments, the polymer or the composition comprising the polymer coats all or a portion of the device. In some embodiments, the coating is applied via a spraying or deposition process. In some embodiments, the coating is applied by dipping or submerging the device into a solution comprising the polymer or the composition comprising the polymer, or starting materials useful in providing the same. In some embodiments, the coating is applied after formation of the polymer. In some embodiments, the polymer forms on a surface of the device or within throughout the entire device or a region of the device. In an example, the device is an absorbent pad that is saturated with a solution comprising a first starting material useful in generating the polymer and a second starting material useful in generating the polymer is subsequently provided to the absorbent pad to promote formation of the polymer throughout the absorbent pad. In some embodiments, the device comprises one or more features useful in promoting or localizing polymerization of starting materials useful in generating a polymer of the present disclosure on and/or within the device. For example, in some embodiments, the device comprises one or more features on the surface of the device for promoting formation of the polymer on the surface of the device, such as one or more protrusions or grids.

In general, the effective amount of the patency maintaining agent and/or one or more therapeutic agents, such as one or more agents effective in promoting hemostasis, will be in the range of from about 0.1 μg per gram of treated tissue to about 500 μg per gram of treated tissue. In specific embodiments it may range from about 0.1 μg per gram of treated tissue to about 200 μg per gram of treated tissue, in still other embodiments it may be from about 100 μg per gram of treated tissue to about 500 μg per gram of treated tissue. These amounts are expressed in terms of local effective concentrations within the treated tissue, and it should be understood that the concentrations will range from a relatively high level immediately adjacent to the device (e.g., applied or implanted device) or composition, to insignificant levels in distant tissues.

A device (e.g., absorbent pad) comprising at least one therapeutic agent will be configured to contain a suitable amount of the at least one therapeutic agent. For example, the device (e.g., absorbent pad) will contain a suitable amount of the at least one therapeutic agent to deliver a sufficient amount of the at least one therapeutic agent to a patient. For therapeutic agents designed to target a microorganism, the suitable amount of the at least one therapeutic agent will be determined by the microorganism(s) targeted. In some embodiments, the device (e.g., absorbent pad) contains from about 1,000 μg to about 50,000 μg of the at least one therapeutic agent. In some embodiments, the device (e.g., absorbent pad) contains between about 1,000 μg to about 5,000 μg about 5,000 μg to about 10,000 about 1,000 μg to about 2,000 about 2,000 μg to about 3,000 μg, about 3,000 μg to about 4,000 μg, about 4,000 μg to about 5,000 μg or more of the at least one therapeutic agent. In some embodiments, the device (e.g., absorbent pad) contains between about 1,000 μg and about 2,000 μg, about 200 μg and about 300 μg, about 300 μg and about 400 μg , about 5,000 μg and about 6,000 μg, about 6,000 μg and about 7,000 μg, about 7,000 μg and about 8,000 μg, about 8,000 μg and about 9,000 μg of the at least one therapeutic agent. In some embodiments, the device (e.g., absorbent pad) contains up to about 200 mg of the at least one therapeutic agent. In some embodiments, the device (e.g., absorbent pad) contains up to about 100 mg of the at least one therapeutic agent. In some embodiments, the device (e.g., absorbent pad) contains between about 1 mg to about 200 mg of the at least one therapeutic agent, such as about 1 mg to about 10 mg, about 10 mg to about 20 mg, about 20 mg to about 30 mg, about 30 mg to about 40 mg, about 40 mg to about 50 mg, about 60 mg to about 70 mg, about 70 mg to about 80 mg, about 80 mg to about 90 mg, or about 90 mg to about 100 mg. In some embodiments, the device (e.g., absorbent pad) contains about 100 mg to about 100 mg, about 110 mg to about 120 mg, about 120 mg to about 130 mg, about 130 mg to about 140 mg, about 140 mg to about 150 mg, about 160 mg to about 170 mg, about 180 mg to about 190 mg, or about 190 mg to about 100 mg of the at least one therapeutic agent. In some embodiments, the device (e.g., absorbent pad) contains about 25 mg, about 50 mg, about 75 mg, about 100 mg, about 125 mg, about 150 mg, about 175 mg, or about 200 mg of the at least one therapeutic agent. A device (e.g., absorbent pad) comprising at least one patency maintaining agent will be configured to contain a suitable amount of the at least one patency maintaining agent. For example, the device (e.g., absorbent pad) will contain a suitable amount of the at least one patency maintaining agent to deliver a sufficient amount of the at least one patency maintaining agent to a patient, and/or to maintain the patency of the device for a proscribed period of time. In some embodiments, the device (e.g., absorbent pad) contains from about 1,000 μg to about 50,000 μg of the at least one patency maintaining agent. In some embodiments, the device (e.g., absorbent pad) contains between about 1,000 μg to about 5,000 μg, about 5,000 μg to about 10,000 μg, about 1,000 μg to about 2,000 μg, about 2,000 μg to about 3,000 μg, about 3,000 μg to about 4,000 μg, about 4,000 μg to about 5,000 μg or more of the at least one patency maintaining agent. In some embodiments, the device (e.g., absorbent pad) contains between about 1,000 μg and about 2,000 μ, about 200 μg and about 300 μg, about 300 μg and about 400 μg, about 5,000 μg and about 6,000 μg, about 6,000 μg and about 7,000 μg, about 7,000 μg and about 8,000 μg, about 8,000 μg and about 9,000 μg of the at least one patency maintaining agent. In some embodiments, the device (e.g., absorbent pad) contains up to about 200 mg of the at least one patency maintaining agent. In some embodiments, the device (e.g., absorbent pad) contains up to about 100 mg of the at least one patency maintaining agent. In some embodiments, the device (e.g., absorbent pad) contains between about 1 mg to about 200 mg of the at least one patency maintaining agent, such as about 1 mg to about 10 mg, about 10 mg to about 20 mg, about 20 mg to about 30 mg, about 30 mg to about 40 mg, about 40 mg to about 50 mg, about 60 mg to about 70 mg, about 70 mg to about 80 mg, about 80 mg to about 90 mg, or about 90 mg to about 100 mg. In some embodiments, the device (e.g., absorbent pad) contains about 100 mg to about 100 mg, about 110 mg to about 120 mg, about 120 mg to about 130 mg, about 130 mg to about 140 mg, about 140 mg to about 150 mg, about 160 mg to about 170 mg, about 180 mg to about 190 mg, or about 190 mg to about 100 mg of the at least one patency maintaining agent. In some embodiments, the device (e.g., absorbent pad) contains about 25 mg, about 50 mg, about 75 mg, about 100 mg, about 125 mg, about 150 mg, about 175 mg, or about 200 mg of the at least one patency maintaining agent.

A device (e.g., absorbent pad) comprising at least one agent for promoting hemostasis (e.g., coagulation agent) will be configured to contain a suitable amount of the at least one agent for promoting hemostasis. For example, the device (e.g., absorbent pad) will contain a suitable amount of the at least one agent for promoting hemostasis to deliver a sufficient amount of the at least one agent for promoting hemostasis to a patient. In some embodiments, the amount of the at least one agent for promoting hemostasis included in a device is selected based on the size and/or position of the wound to be treated. In some embodiments, the device (e.g., absorbent pad) contains from about 1,000 μg to about 50,000 μg of the at least one agent for promoting hemostasis. In some embodiments, the device (e.g., absorbent pad) contains between about 1,000 μg to about 5,000 μg, about 5,000 μg to about 10,000 μg, about 1,000 μg to about 2,000 μg, about 2,000 μg to about 3,000 μg, about 3,000 μg to about 4,000 μg, about 4,000 μg to about 5,000 μg or more of the at least one agent for promoting hemostasis. In some embodiments, the device (e.g., absorbent pad) contains between about 1,000 μg and about 2,000 μg, about 200 μg and about 300 μg, about 300 μg and about 400 μg, about 5,000 μg and about 6,000 μg, about 6,000 μg and about 7,000 μg, about 7,000 μg and about 8,000 μg, about 8,000 μg and about 9,000 μg of the at least one agent for promoting hemostasis. In some embodiments, the device (e.g., absorbent pad) contains up to about 200 mg of the at least one agent for promoting hemostasis. In some embodiments, the device (e.g., absorbent pad) contains up to about 100 mg of the at least one agent for promoting hemostasis. In some embodiments, the device (e.g., absorbent pad) contains between about 1 mg to about 200 mg of the at least one agent for promoting hemostasis, such as about 1 mg to about 10 mg, about 10 mg to about 20 mg, about 20 mg to about 30 mg, about 30 mg to about 40 mg, about 40 mg to about 50 mg, about 60 mg to about 70 mg, about 70 mg to about 80 mg, about 80 mg to about 90 mg, or about 90 mg to about 100 mg. In some embodiments, the device (e.g., absorbent pad) contains about 100 mg to about 100 mg, about 110 mg to about 120 mg, about 120 mg to about 130 mg, about 130 mg to about 140 mg, about 140 mg to about 150 mg, about 160 mg to about 170 mg, about 180 mg to about 190 mg, or about 190 mg to about 100 mg of the at least one agent for promoting hemostasis. In some embodiments, the device (e.g., absorbent pad) contains about 25 mg, about 50 mg, about 75 mg, about 100 mg, about 125 mg, about 150 mg, about 175 mg, or about 200 mg of the at least one agent for promoting hemostasis.

In some embodiments, the device (e.g., absorbent pad) contains up to about 200 mg of the at least one therapeutic agent, patency maintaining agent, and/or agent for promoting hemostasis. In some embodiments, the device (e.g., absorbent pad) contains up to about 100 mg of the at least one therapeutic agent, patency maintaining agent, and/or agent for promoting hemostasis. In some embodiments, the device (e.g., absorbent pad) contains between about 1 mg to about 200 mg of the at least one therapeutic agent, patency maintaining agent, and/or agent for promoting hemostasis, such as about 1 mg to about 10 mg, about 10 mg to about 20 mg, about 20 mg to about 30 mg, about 30 mg to about 40 mg, about 40 mg to about 50 mg, about 60 mg to about 70 mg, about 70 mg to about 80 mg, about 80 mg to about 90 mg, or about 90 mg to about 100 mg. In some embodiments, the device (e.g., absorbent pad) contains about 100 mg to about 100 mg, about 110 mg to about 120 mg, about 120 mg to about 130 mg, about 130 mg to about 140 mg, about 140 mg to about 150 mg, about 160 mg to about 170 mg, about 180 mg to about 190 mg, or about 190 mg to about 100 mg of the at least one therapeutic agent, patency maintaining agent, and/or agent for promoting hemostasis. In some embodiments, the device (e.g., absorbent pad) contains about 25 mg, about 50 mg, about 75 mg, about 100 mg, about 125 mg, about 150 mg, about 175 mg, or about 200 mg of the at least one therapeutic agent, patency maintaining agent, and/or agent for promoting hemostasis.

In some embodiments, a device (e.g., absorbent pad) provides release in a controlled fashion for a sustained period of time, i.e., serves as a sustained-release device. Various elements of the device composition, physical characteristics, placement location, and the amount and composition of a therapeutic agent, patency maintaining agent, and/or agent for promoting hemostasis work in combination to produce the desired release profile of the therapeutic agent, patency maintaining agent, and/or agent for promoting hemostasis.

In some embodiments, the composition and/or device (e.g., absorbent pad) is configured for delivery of an agent (e.g., a patency maintaining agent and/or therapeutic agent, such as an agent for promoting hemostasis) for a suitable period of time. In some embodiments, composition and/or device (e.g., absorbent pad) is configured for delivery of the at least one patency maintaining agent and/or therapeutic agent (e.g., an agent for promoting hemostasis) for at least 1 day, at least about 3 days, at least about five days, at least about one week, at least about two weeks, at least about three weeks, or at least about four weeks. In some embodiments, the composition and/or device (e.g., absorbent pad) is capable of delivering the at least one patency maintaining agent and/or therapeutic agent (e.g., an agent for promoting hemostasis) for at least one month, at least two months, at least three months, at least four months, at least five months, or at least six months or more. In some embodiments, the composition and/or device (e.g., absorbent pad) delivers the at least one patency maintaining agent and/or therapeutic agent (e.g., an agent for promoting hemostasis) for a period of about 2 to about 4 weeks and then degrades.

In some embodiments, the composition or device (e.g., absorbent pad) degrades within six weeks of placement (e.g., implantation) or application. In some embodiments, about 25% to about 50% of an agent included in the composition or device (e.g., a patency maintaining agent, therapeutic agent, and/or agent for promoting hemostasis) is released from the device (e.g., absorbent pad) during the first 7 days following implantation or application. In embodiments, from about 50% to about 100%, such as from about 50% to about 90%, of the agent is released from the device (e.g., absorbent pad) during the first 7 days following implantation or application.

In some embodiments, one or more agents (e.g., one or more patency maintaining agents, therapeutic agents, and/or agents for promoting hemostasis) delivered by means of a composition or device (e.g., absorbent pad) are co-administered with one or more additional agents delivered by another mode of administration, for example, orally or intravenously. Co-administration encompasses simultaneous administration as well as administration by the additional mode before or after administration by the composition or device (e.g., absorbent pad).

In some embodiments, the polymer (e.g., biodegradable polymer) of a composition or device surrounds a core of one or more agents (e.g., one or more patency maintaining agents, therapeutic agents, or agents for promoting hemostasis). In another embodiment, the one or more agents are dispersed within the polymer (e.g., biodegradable polymer) to form a biodegradable mono-lithic device. Degradation of the polymer (e.g., biodegradable polymer) and/or biodegradable mono-lithic device can occur as surface and/or bulk processes. In some embodiments, degradation of the polymer (e.g., biodegradable polymer) and/or biodegradable mono-lithic device comprises breaking of covalent bonds.

In some embodiments, a device is configured for placement in or on a given region of a body, such as a region of a body that requires treatment. For example, in some embodiments, a device is configured for placement in a nasal passage. In other embodiments, the device is configured for placement in a region of the ear. In some embodiments, the device is configured for placement on a wound on an area of skin. In some embodiments, the size and shape of the device are selected based on the region of the body where the device will be placed. In an example, the size and shape of the device are selected for placement in a nasal passage (e.g., a nasal passage of an adult or a nasal passage of a child). In another example, the size and shape of the device are selected for placement in the ear (e.g., an ear of an adult or an ear of a child). In some embodiments, the size and shape of the device are selected based on the size (e.g., gender and/or age) of an individual that will be treated with the device.

In some embodiments, the composition and/or device (e.g., absorbent pad) is useful in treating a nasal condition by maintaining patency of an aperture formed therein for a certain period of time. In some embodiments, the device elutes or releases a patency maintaining agent but does not elute or release a therapeutic agent.

In some embodiments, the device (e.g., absorbent pad) elutes or releases a patency maintaining agent and at least one therapeutic agent (e.g., as described elsewhere herein). In some embodiments, the at least one therapeutic agent is selected from the group consisting of a corticosteroid, a decongestant, an immunotherapeutic agent, an adrenocorticoid, an analgesic agent, an analgesic adjunct, an analgesic-anesthetic, an anesthetic, an antibiotic, an antibacterial agent, an anti-infective agent, an antibiotic therapy adjunct, an antidote, an anti-emetic agent, an anti-fungal agent, an anti-inflammatory agent, an anti-vertigo agent, an anti-viral agent, a biological response modifier, a cytotoxic agent, a diagnostic aid, an immunizing agent, an immunomodulator, proteins, and peptides.

In some embodiments, the composition and/or device (e.g., absorbent pad) is useful in treating a nasal condition without administration of antibiotics. In some embodiments, such a composition and/or device may elute one or more therapeutic agents other than an antibiotic.

Advantageously, the methods of the present disclosure permit treatment of a nasal condition and/or cessation of bleeding in a nasal passage while keeping the nasal passage open for breathing and/or discharge. The methods of the present disclosure avoid the limitations of absorbent packing materials that mechanically stop blood flow that can be uncomfortable, can result in infection, and block air passages.

The healing process of a wound following surgical incision and/or removal of a feature such as a nasal polyp or other growth is intricate and in some cases displays several stages, including an inflammatory phase and tissue proliferation periods. Similarly, other wounds induced by other means (severing, slicing, stabbing, etc.) have variable healing processes dependent upon the type, severity, and location of the wound. Accordingly, the period of time over which a composition and/or device of the present disclosure is used varies based on the wound type, location, and/or surgical process.

In some embodiments, a device (e.g., absorbent pad) is maintained on a wound for about 10 minutes, about 20 minutes, about 30 minutes, about 40 minutes, about 50 minutes, about 60 minutes, about 90 minutes, about 120 minutes, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, about 24 hours, about 1.5 days, about 2 days, about 4 days, about 1 week, or longer. In some embodiments, a device (e.g., absorbent pad) is maintained on a wound for longer than about 1 week, such as about 2 weeks, about 3 weeks, about 1 month, or longer. In some embodiments, a device (e.g., absorbent pad) is maintained on a wound until all or a portion of a therapeutic agent (e.g., as described herein) is delivered to a treatment site. In some embodiments, all or a portion of a composition or device (e.g., absorbent pad) applied to a treatment site dissolves or otherwise changes form over a period of time, such as over several days, weeks, or months.

In some embodiments, the methods of the present disclosure prevent or delay the healing process of a wound. In some embodiments, a composition or device (e.g., absorbent pad) used to promote hemostasis (e.g., at a site of a nasal wound such as a site from which a nasal polyp was removed) reduces or eliminates bleeding at a treatment site but does not heal the wound. In some embodiments, a composition or device (e.g., absorbent pad) used to promote hemostasis (e.g., at a site of a nasal wound such as a site from which a nasal polyp was removed) reduces or eliminates bleeding at a treatment site but does not heal immediately the wound. In some such embodiments, a therapeutic agent (e.g., as described herein) is delivered by the composition or device (e.g., absorbent pad) to heal the wound (e.g., promote tissue proliferation, reduce inflammation, or otherwise promote healing of the treatment site) over a period of time, such as over several hours, days, or weeks. In some embodiments, the methods of the present disclosure inhibit cell growth at a wound site.

In some embodiments, a placement guide or tool is used to assist in applying or implanting the device (e.g., absorbent pad). In some embodiments, applying the device involves removing an applicator such as a contact paper to provide the device to a treatment site. In an example, the device is an absorbent pad having an adhesive component, which adhesive component is contacted with a contact paper prior to application of the device to the treatment site and which contact paper is removed to expose the adhesive component to the treatment site to facilitate a connection between the device and the treatment site. In another example, the device is an absorbent pad to which a polymer (e.g., a biodegradable polymer) of the present disclosure is applied, and the polymer adheres the device to the treatment site. In another example, the device is an absorbent pad that is applied to a treatment site to which a polymer (e.g., a biodegradable polymer) has previously been applied, and the polymer adheres the device to the treatment site.

In some embodiments, the device is provided to the subject immediately following a surgical procedure such as a surgical procedure to remove one or more nasal polyps. In some embodiments, a device (e.g., absorbent pad) is provided to (e.g., implanted in or applied to) a subject while the subject is under general anesthesia (e.g., a young child) or local anesthesia (e.g., an adult). When utilized, general anesthesia is induced via mask ventilation. When location anesthesia is used (e.g., a spray or cream), it should be applied about 30 minutes prior to the procedure. The subject is generally supine, with the head positioned square to bed and then rotated laterally, with the nose about 30-45° away from vertical. The treatment area is cleaned (e.g., washed) before the device is applied or implanted.

In some embodiments, a composition comprising a polymer (e.g., biodegradable polymer) of the present disclosure is a solution. In some embodiments, a composition comprising a polymer (e.g., biodegradable polymer) of the present disclosure is or comprises a gel. In some embodiments, a composition is directly applied to a treatment site (e.g., in a nasal passage). In an example, the composition is or comprises a gel and the gel is applied directly to the treatment site by, for example. In another example, the composition is or comprises a solution and the solution is applied to the treatment site by spraying. In such an example, the polymer hardens and/or solidifies after application of the composition to the treatment site.

In some embodiments, a composition and/or device comprising a polymer (e.g., biodegradable polymer) of the present disclosure is useful in treatment of wounds in an area of the body other than a passage (e.g., a nasal passage). For example, in some embodiments, the composition or device is useful in treatment of wounds on open areas of skin, e.g., for promoting hemostasis at a wound site on an open area of skin.

### Methods of Manufacture

The present disclosure further provides methods of manufacturing the devices (e.g., absorbent pads) described herein. The devices of the present disclosure can be manufactured by any suitable method.

In some embodiments, the device (e.g., absorbent pad) is manufactured by molding the device (e.g., by injection molding or another method) and then providing a polymer of the present disclosure to the device. In some embodiments, the polymer is formed on or within the device. In other embodiments, the polymer is provided (e.g., applied) to a surface of the device.

### Systems and Kits

The present disclosure further provides systems comprising a composition or device comprising a polymer (e.g., a biodegradable polymer) described herein. In some embodiments, the system includes a device (e.g., absorbent pad) as well as one or more components selected from the group consisting of an operative microscope, a speculum set, a knife or blade, a suction setup, and combinations thereof.

The present disclosure further provides kits comprising a composition or device comprising a polymer (e.g., a biodegradable polymer) described herein. In some embodiments, the kit includes a device (e.g., an absorbent pad) as well as one more components useful in connection with the same.

In some embodiments, the kit comprises a first starting material useful in generating the polymer and a second starting material useful in generating the polymer. In some embodiments, the first starting material is a Michael acceptor. In some embodiments, the second starting material is a Michael donor. In some embodiments, the kit further comprises a third starting material. In some embodiments, the first starting material and the second starting material are provided in separate containers. In some embodiments, the first starting material and/or the second starting material are provided in a syringe. In some embodiments, the kit further comprises instructions for preparing the polymer from the first starting material and the second starting material. In some embodiments, such instructions comprise instructions to combine the first starting material with the second starting material and to apply the resultant solution (e.g., a solution comprising the polymer or a precursor to the polymer) to the treatment site within, for example, about 1 minute, about 5 minutes, about 10 minutes, about 15 minutes, about 20 minutes, about 30 minutes, about 45 minutes, about 1 hour, or about 2 hours. In some embodiments, such instructions comprise instructions to apply the resultant polymer or solution comprising the polymer or a precursor thereof to the treatment site by spraying the material on the treatment site.

### EXAMPLES

### Example 1

### Preparation of Materials

A stirring solution of trimethylolpropane tris(3-mercaptopropionate) (1 ml) (Scheme 7) in 5 ml sodium hydrogen phosphate buffer solution (pH 9.8) was mixed in a Falcon tube with trimethylolpropane ethoxylate triacrylate (0.75 ml) and heated to 45° C. in a water bath for 24 hours. No reaction was observed. To this solution was added acetonitrile (1 ml), after which a polymer was formed in 30 minutes. The hard and brittle polymer was removed from the Falcon tube. A 0.2 g sample of the polymer was placed in 20 ml buffer at pH 8.00 in a 50° C. oven to study the degradation characteristics of the material. The material did not degrade or dissolve even after 1.5 years under the conditions. Thus the degradation time is more than 1.5 years at pH 8.0.

In another embodiment to this reaction, the solvent was changed to acetonitrile (1 ml) and a polymer was formed within 30 minutes.

### Example 2

### Preparation of Materials

A stirring 4-Arm PEG-10K-Acrylate (10K MW, 0.5 gm.) solution in water (0.75 ml) was vortex mixed in a 20 ml Falcon tube with 0.25 ml sodium hydrogen phosphate buffer solution (pH 9.8) at ambient temperature. To this mixture was added 4,4′-bis(mercaptomethyl)biphenyl solution (0.18 g) and the mixture was placed in 45° C. water bath. A polymer was formed in about 2-4 minutes. The polymer is bouncy and absorbs less than 10% water by weight.

### Example 3

### Preparation of Material and Device

A stirring 4-Arm PEG-Acrylate (10K MW, 0.5 g) solution in water (0.75 ml) was vortex mixed in a 20 ml Falcon tube with 0.25 ml sodium hydrogen phosphate buffer solution (pH 9.80) at ambient temperature. To this mixture was added trimethylolpropane tris(mercapto propionate) solution (0.40 g) after mixing for about 20 seconds, a polymer was formed in about 60-90 seconds at ambient temperature.

### Example 4

### Preparation of Material and Device—at Lower pH and with Two Acrylate Monomers

A stirring 4-Arm PEG-Acrylate (10K MW, 0.25 gm) solution in water (0.50 ml) was vortex mixed with 0.25 ml sodium hydrogen phosphate buffer solution (pH 8.0) at ambient temperature in a 20 ml Falcon tube. To this mixture was added trimethylolpropane tris(mercapto propionate (0.08 g) and trimethylol propane ethoxylate triacrylate (0.06 g). The reactive mixture was vortex mixed for about 30 seconds. It takes about 3-4 minutes to form the gel. The degradation time was 20 days at pH 8.00.

### Example 5

### Preparation of Material and Device

A stirring 4-Arm PEG-Acrylate (10K MW, 0.25 gm) solution in water (0.50 ml) was vortex mixed with 0.25 ml pH sodium hydrogen phosphate buffer solution (pH 8.0) at ambient temperature. To this mixture was added trimethylolpropane tris(mercapto propionate (0.08 g) and trimethylol propane ethoxylate triacrylate (0.06 g). The reactive mixture was vortex mixed for about 30 seconds and poured into a mold of the device shape desired and allowed to gel inside the mold. It takes about 3-4 minutes to form the gel. After removing the mold, the desired parts are obtained and characterized. In some embodiments, the device shape is a rectangular, ovular, or circular pad.

The mold was placed in the physiological buffer for the degradation study. The degradation time was 20 days.

### Example 6

### Preparation of Material and Device With Improved Monomer Solubility—Lower pH

A 4-Arm PEG-Acrylate (10K MW, 0.1 g) powder was mixed with 4 ml sodium hydrogen phosphate buffer solution (0.01 molar, pH 7.4) in a 20 ml Falcon tube at ambient temperature. To this mixture was added 4-arm-PEG-10K-Thiol (0.1 g) and vortex mixed for 2 minutes. It appears that there are some undissolved powders floating. The mixture was placed at ambient temperature to react. The gel formation observed at about 7 minutes when a blob was formed and separated from the liquid. The gel formation continued until to about 15 minutes when the entire liquid gelled.

### Example 7

### Preparation of Material and Device—Lyophilized Gel

A 4-Arm PEG-Acrylate (10K MW, 0.2 g) powder was mixed with 8 ml sodium hydrogen phosphate buffer solution (0.01 molar, pH 7.4) in a 20 ml Falcon tube at ambient temperature. To this mixture was added 4-arm-PEG-10K-Thiol (0.2 g) and vortex mixed for 2 minutes. It appears that there are some un-dissolved powders floating. The reactive mixture was poured in 3 steel trays (about 1″×1″) of about equal volumes and allowed to react for about 8 minutes. After this time, the trays were placed at −20 ° C. in a lyophilyzer for about 20 minutes to freeze completely and then freeze dried for 20 hours under vacuum of 0.01 mTorr and then warmed up to the room temperature. The polymers were removed from the trays. The polymer looks fluffy but does absorb liquid quite quickly (in less than 1-2 seconds on contact with water). The polymer swells but is not bouncy in the dry and wet state. When compressed in dry state, it stays compressed and it takes a long time (1-2 days) for it to get to the original volume.

### Example 8

### Preparation of Material and Device—Both Monomers Water Soluble

A 4-Arm PEG-Acrylate (10K MW, 0.1 g) powder was mixed with 4 ml sodium hydrogen phosphate buffer solution (0.01 molar, pH 9.8) in a 20 ml Falcon tube at ambient temperature. To this mixture was added 4-arm-PEG-10K-Thiol (0.1 g) and vortex mixed for 30 seconds. It appears that there are still some undissolved powders floating. The mixture was placed at ambient temperature to react to form the gel. The gel formation observed at about 50-70 seconds with signs of strings formation and separation of the polymer from the remaining liquids. The polymer formation is not uniform and homogeneous. It is likely due to the poor solubility of the 4-Arm PEG-10K Acrylate in the aqueous solution.

### Example 9

### Evaluation of Bonding Ability of the Polymer with Beef Steak

Powder mixtures of 4-Arm PEG-Acrylate (10K MW, 0.1 g) and 4-arm-PEG-10K-Thiol (0.1 g) were paced inside a 10 ml syringe. In another syringe 4 ml sodium hydrogen phosphate buffer solution (0.01 molar, pH 9.8) was measured and placed. The front ends of the two syringes were connected with a Luer lock connector and the contents were mixed by pressing the syringe plungers back and forth for about 20 seconds. The reactive mixture was carefully dispensed over a selected area of a fresh beef steak (to simulate an open wound) and the gel formation was observed. It took about 50-70 seconds to form the gel over the steak surface.

The bonding of the polymer to the steak surface was evaluated by careful attempts to remove the polymer from the steak surface. The polymer peeled of as a film from the steak surface. Apparently the polymer did not bond well.

### Example 10

### Evaluation of Bonding Ability of the Polymer with Beef Steak and a Longer Gel Time Polymer (Lower pH, 7.86)

Powder mixtures of 4-Arm PEG-acrylate (10K MW, 0.1 g) and 4-arm-PEG-10K-thiol (0.1 g) were placed inside a 10 ml syringe. In another syringe 4 ml sodium hydrogen phosphate buffer solution (0.01 molar, pH 7.86) was measured and placed. The front ends of the two syringes were connected with a Luer lock connector and the contents were mixed by pressing the syringe plungers back and forth for about 20 seconds. The reactive mixture was carefully dispensed over a selected area of the fresh beef steak and the gel formation was observed. It took about 5-6 minutes to form the gel over the steak surface.

The bonding of the polymer to the steak surface was evaluated by careful attempts to remove the polymer from the steak surface. The bonding was not strong, perhaps the polymer was non homogenous and the bonding was observed at some places but peeled off from other locations.

### Example 11

### Evaluation of Bonding Ability of the Polymer with Beef Steak and a Longer Gel Time Polymer (Lower pH 7.86, Higher Concentration)

Powder mixtures of 4-Arm PEG-acrylate (10K MW, 0.1 g) and 4-arm-PEG-10K-thiol (0.1 g) were placed inside a 10 ml syringe. In another syringe 2 ml sodium hydrogen phosphate buffer solution (0.01 molar, pH 7.86) was measured and placed. The front ends of the two syringes were connected with a Luer lock connector and the contents were mixed by pressing the syringe plungers back and forth for about 20 seconds. The reactive mixture was carefully dispensed over a selected area of the fresh beef steak and the gel formation was observed. It took about 5-6 minutes to form the gel over the steak surface.

The bonding of the polymer to the steak surface was evaluated by careful attempts to remove the polymer from the steak surface. The bonding was strong and uniform across the entire surface area of the steak and the polymer did not peel off from the surface. During further attempted removal, the polymer broke but did not peel off

The degradation time of the polymer at 43° C. oven temperature was 11 days at pH 7.86

### Example 12

### Longer Gel Time Polymer (Higher Concentration, Lower pH 7.6), 4-arm-PEG20K-Acrylate

Powder mixtures of 4-Arm PEG-20K-acrylate_(0.2 g) and 4-arm-PEG-10K-thiol (0.1 g) were placed inside a 10 ml syringe (Scheme 6). In another syringe 2.4 ml sodium hydrogen phosphate buffer solution (0.01 molar, pH 7.6) was measured and placed. The front ends of the two syringes were connected with a Luer lock connector and the contents were mixed by pressing the syringe plungers back and forth for about 20 seconds. The reactive mixture was carefully dispensed in a weighing boat. It took about 16 minutes to form the gel. The gel is bouncy and firm. The degradation time of the polymer at 46° C. oven temperature was 7 days at pH 7.86.

The degradation times and of selected formulations at various pH's are shown in the Table below.

TABLE 1
Details of polymer formulations.

| | | | | Water | |
|---|---|---|---|---|---|
| | | Gel Time | Degradation | bath | Degradation |
| | Polymer | (min) | Buffer pH | Temp | time |
| 1 | SA-01-42 | 16.0 min | 7.86 | 46° C. | 5-7 days |
| | 4-Arm PEG-20K acrylate and | | | | |
| | 4-arm-PEG-10K-thiol | | | | |
| 2 | SA-01-43-A | 6.46 min | 7.6 | 37° C. | 14 days |
| | 4-Arm PEG-10K acrylate and | | | | |
| | 4-arm-PEG-20K-thiol | | | | |
| 3 | SA-01-43-B | 6.46 min | 7.6 | 47° C. | 7 days |
| | 4-Arm PEG-20K acrylate and | | | | |
| | 4-arm-PEG-10K-thiol | | | | |
| 4 | SA-01-43 | 5.40 min | 7.6 | 37° C. | 20 days |
| | 4-Arm PEG-10K acrylate and | | | | |
| | 4-arm-PEG-10K-thiol (This | | | | |
| | polymer had a green dye added | | | | |
| | to it) | | | | |

### Example 13

### Evaluation of Bonding Ability of the Polymer In Mouse Abdominal Model

Powder mixtures of 4-Arm PEG-10K acrylate (0.1 g) and 4-arm-PEG-10K-thiol (0.1 g) were placed in a 10 ml syringe. In another syringe 2 ml sodium hydrogen phosphate buffer solution (0.01 molar, pH 7.86) was measured and placed. The two syringes were connected with a Luer lock connector and the contents were mixed by pressing the plungers back and forth for about 20 seconds. The reactive mixture was carefully dispensed over a surgically cut and exposed mouse abdomen and skin and the gel formation was observed. It took about 5-6 minutes to form the gel over the organs and the skin surface. The bonding of the polymer to the organs surface was evaluated by careful attempts to remove the polymer from the surface. The bonding was strong and uniform across the entire surface area and the polymer did not peel off from the surface. During further attempted removal, the polymer broke but did not peel off.

### Example 14

### Evaluation of Bonding Ability of the Polymer in Pig Mucosa Model

Powder mixtures of 4-Arm PEG-20K acrylate (0.06 g) and 4-arm-PEG-10K-thiol (0.03 g) were placed in a 10 ml syringe. In another syringe 0.6 ml sodium hydrogen phosphate buffer solution (0.01 molar, pH 7.86) containing a small amount of green food dye (for visualization purposes) was measured and placed. The two syringes were connected with a Luer lock connector and the contents were mixed by pressing the plungers back and forth for about 20 seconds. The reactive mixture was carefully dispensed over freshly obtained pig nostrils. It took about 5-6 minutes to form the gel over the pig nostrils mucosa. The bonding of the polymer to the pig nostril was evaluated by careful attempts to remove the polymer from the surface. The bonding was strong and uniform across the entire surface area and the polymer did not peel off from the surface. During further attempted removal, the polymer broke but did not peel off.

### Example 15

### Gel Formation Using Soluble 8-Arm-PEG20k Acrylate

Powder mixtures of 8-Arm PEG-20K acrylate (0.12 g) and 4-arm-PEG-10K-thiol (0.03 g) were placed in a 10 ml syringe. In another syringe 2.0 ml sodium hydrogen phosphate buffer solution (0.01 molar, pH 7.86) was measured and placed. The two syringes were connected with a Luer lock connector and the contents were mixed by pressing the plungers back and forth for about 20 seconds. The final contents were pushed into one syringe and the gel time was measured. The gel time was ˜18 min.

The polymer degradation time at 7.86 pH/ 48° C. was 5 days and at 7.6 pH/48° C. the degradation time was 7 days.

### Example 16

### Gel Formation Using Soluble 4-Arm-PEG20k Acrylate

Powder mixtures of 4-Arm PEG-20K acrylate (0.06 g) and 4-arm-PEG-10K-thiol (0.03 g) were placed in a 10 ml syringe. In another syringe 0.6 ml sodium hydrogen phosphate buffer solution (0.01 molar, pH 7.86) was measured and placed. The two syringes were connected with a Luer lock connector and the contents were mixed by pressing the plungers back and forth for about 20 seconds. The final contents were pushed into one syringe and the gel time was measured. The gel time was ˜8 min.

### Example 17

### Gel Time Reduction at a Constant pH

Powder mixtures of 4-Arm PEG-20K acrylate (0.05 g) and 4-arm-PEG-10K-thiol (0.025 g) were placed in a 10 ml syringe. In another syringe 0.6 ml sodium hydrogen phosphate buffer solution (0.01 molar, pH 7.86, containing 0.5% by weight methyl cellulose, from Sigma Aldrich, M0512, viscosity 4000 cP for 2% in DI) was measured and placed. The two syringes were connected with a Luer lock connector and the contents were mixed by pressing the plungers back and forth for about 20 seconds. The final contents were pushed into one syringe and the gel time was measured. The gel time was ˜9 min.

### Example 18

### Gel Time Reduction at a Constant pH

Powder mixtures of 4-Arm PEG-20K acrylate (0.05 g) and 4-arm-PEG-10K-thiol (0.025 g) were placed in a 10 ml syringe. In another syringe 0.6 ml sodium hydrogen phosphate buffer solution (0.01 molar, pH 7.86, containing 0.25% by weight hydroxyl propyl cellulose, from Sigma Aldrich, 435007, average M_{w} 80,000, Mn 10,000) was measured and placed. The two syringes were connected with a Luer lock connector and the contents were mixed by pressing the plungers back and forth for about 20 seconds. The final contents were pushed into one syringe and the gel time was measured. The gel time was ˜2.5 minutes. The samples were placed in for degradation study. The degradation time was 8 days at pH 7.86 at 47° C. and was 14 days at pH 37° C. The degradation time of the polymer at 37° C. in pH 7.4 is 24 days.

### Example 19

### Degradation of Measurements of the Polymer

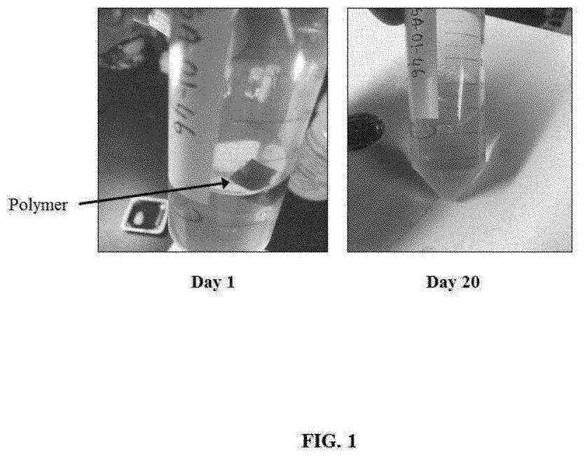

A piece of the polymer sample (about 0.5 g) was placed in a 20 ml buffered saline at 37° C. water bath. The presence of the polymer was observed over time. The degradation time till the total dissolution of the polymer is measured. The degradation time is found to be 2-8 weeks depending on the formulations used. FIG. 1 shows degradation of a polymer over 20 days.

### Example 20

### Degradation Time and Drug Elution

A mold piece manufactured per example 5 is placed in a 2 ml buffered saline at 37° C. water bath. The entire 2 ml saline is removed daily from the sample and a new 2 ml saline is added. The process is repeated daily till the device is completely dissolved. The saline sample with drug is analyzed by HPLC, GC to establish the elution rate.

### Example 21

### Investigation of Controlled Bleeding in Mice

### Objective

The primary objective of the study was to examine the ability of a preparation of the present disclosure to control/stop bleeding in mice. The secondary objective of this study was to compare two different preparations for their ability to control/stop bleeding and select one preparation for future studies.

### Materials

Polymers were prepared according to the Examples above.

### Methods

### Animal Model

Albino mice (10) weighing approximately (25 g) were used in this experiment.

A total of 10 mice were used in this study. The plan was to use 4 mice for testing the two preparations referred to as Preparation 1 and Preparation 2 respectively, and compare their ability to gel in-time and to stop/control bleeding following the incision in the tail; two mice were used as controls. A similar experimental procedure was followed for all the mice.

Mice were anesthetized appropriately with ether. An incision was made in the tail approximately 3 cm from the tail base. Bleeding stop time was determined by measuring the elapsed time from the time of application of the preparation. For the control mice, blood from incised tail was blotted out periodically using Kimwipes. Time taken for bleeding to stop completely was recorded.

The gelling time for the two preparations (Preparation 1 and Preparation 2) were determined in advance of the mice experiment. Each fluid preparation was delivered to the incision site approximately 30 seconds prior to the anticipated gelling time for both preparations.

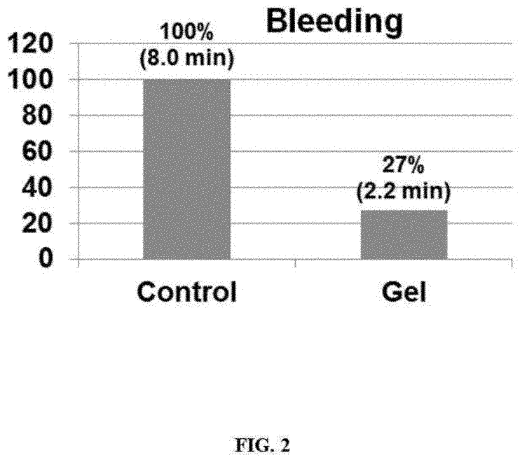

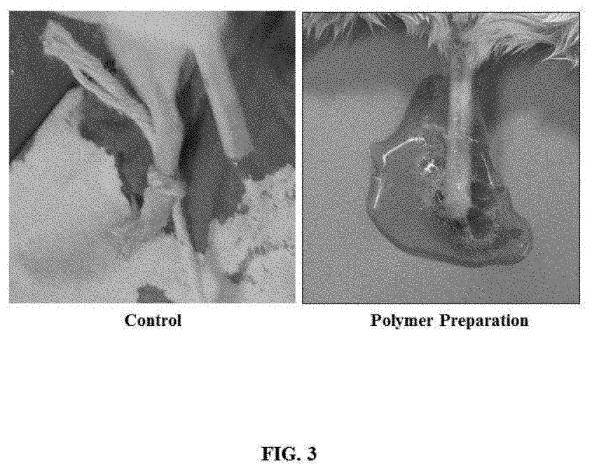

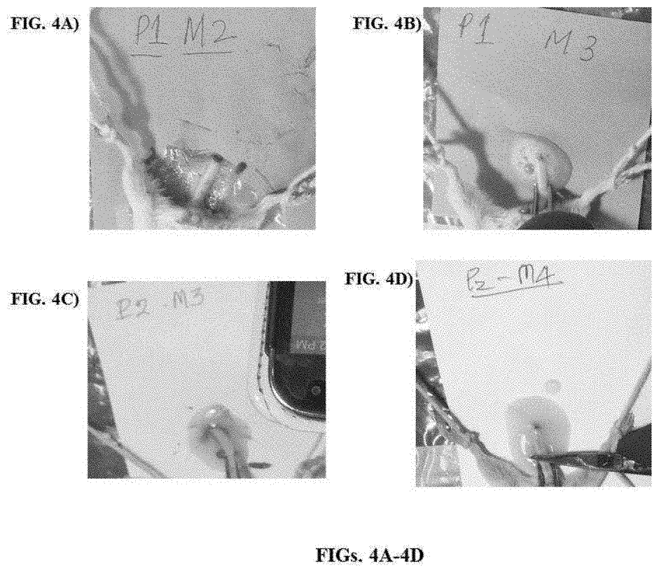

Immediately after tail incision, a few drops of Preparation 1 were delivered directly on the incision site of the tail. Typically, the preparations gelled within 30 seconds of application to the tail. Time taken for the bleeding to stop was recorded. A total of 4 mice were used for testing each of the two Preparations (#1 and #2). Results are presented in Table 2 and FIGS. 2, 3, and 4A-4D.

### Results

The results are shown in Table 2. Preparation #1 was able to control bleeding in mice in about 10% of the time of the untreated, i.e., control. Preparation #2 was able to control bleeding in 9.6% of the time of the untreated, i.e., control. Thus, the results of the two preparations were similar and consistent in their ability to control bleeding.

TABLE 2
Bleeding Control Times

| | | | Time in Minutes | | | |
|---|---|---|---|---|---|---|
| Mouse | Mouse | | Incision | Bleeding Stop | Total Bleeding | Difference |
| No | ID | Treatment | Time (Min) | Time (Min) | Time* (min) | From Control % |
| 1 | 1 | Control | 0 | 14.00 | 14.00 | 100.0 |
| 4 | P1-M1 | Preparation 1 | 1.30 | 3.10 | 1.40 | 10.0 |
| 5 | P1-M2 | Preparation 1 | 1.20 | 3.03 | 1.43 | 10.2 |
| 6 | P1-M3 | Preparation 1 | 1.30 | 3.00 | 1.30 | 9.3 |
| 9 | P2-M3 | Preparation 2 | 2.10 | 3.45 | 1.35 | 9.6 |
| 10 | P2-M4 | Preparation 2 | 2.10 | 3.45 | 1.35 | 9.6 |

### Conclusion

The preparations were effective in controlling bleeding in mice in our lab experiments detailed in this report. Both Preparations, 1 and 2, were effective in stopping bleeding in 10% of the time compared to the untreated control. There was no significant difference between the two preparations.

### Example 22

### Preparation of Materials

A stirring 4,4′-Bis(mercaptomethyl)biphenyl solution (1.44 g) (scheme 1) in ethanol/water (50/50, 5 ml) at ambient temperature was pH adjusted to 7.8 by adding glycylglycine solution. To this solution was added pentaerythritol tetraacrylate (1.05 gm.). The reactive mixture is poured into a desired surface mimicking wound and allowed to gel. It takes about 1-5 minutes to form the gel.

### Example 23

### Preparation of Materials

A stirring 4,4′-bis(mercaptomethyl)biphenyl solution (1.44 g) (Scheme 1) in ethanol/water (50/50, 10 ml) at ambient temperature was pH adjusted to 7.8 by adding glycylglycine solution. To this solution was added 4-Arm PEG-Acrylate (2K MW), powder (6 gm.). The reactive mixture is poured into a desired surface mimicking wound and allowed to gel. It takes about 1-5 minutes to form the gel.

### Example 24

### Preparation of Materials

A stirring 4,4′-bispheno1-4,4′-dithiol solution (1.31 g) (Scheme 2) in ethanol/water (50/50, 10 ml) at ambient temperature was pH adjusted to 7.8 by adding glycyl glycine solution. To this solution was added 4-arm PEG-acrylate (2K MW), powder (6 gm.). The reactive mixture is poured into a desired surface mimicking wound and allowed to gel. It takes about 1-5 minutes to form the gel.

### Example 25

### Preparation of Materials

A stirring glycidyl ether solution (1.44 g) (Scheme 3) in ethanol/water (50/50, 5 ml) at ambient temperature was pH adjusted to 7.8 by adding buffered saline solution. To this solution was added PEG amine and PEG amine ester. The reactive mixture is poured into a desired surface mimicking wound and allowed to gel. It takes about 1-5 minutes to form the gel.

### Example 26

### Preparation of Materials

A stirring 4,4′-Bis(mercaptomethyl)biphenyl solution (1.44 g) (Scheme 2) in ethanol/water (50/50, 5 ml) at ambient temperature was pH adjusted to 7.8 by adding glycylglycine solution. To this solution was added pentaerythritol tetraacrylate (1.05 gm) and KB-R7885 (0.4 gm) or steroids (0.4 gm) etc. The reactive mixture is poured into a desired surface mimicking wound and allowed to gel. It takes about 1-5 minutes to form the gel.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of promoting hemostasis in an individual in need thereof, comprising administering to the individual a composition comprising a biodegradable polymer,
wherein the biodegradable polymer is generated by combining:
(a) a PEG-thiol compound of Formula: 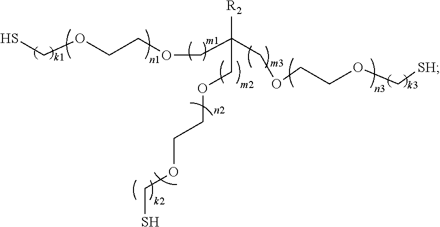
and
(b) a PEG-acrylate compound of Formula: 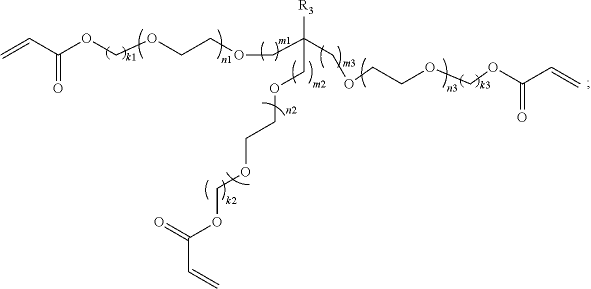
wherein:
R₂is 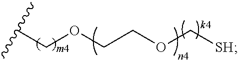
R₃ is 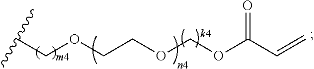
n₁, n₂, n₃, and n₄ are independently 40-60; and
m₁, m₂, m₃, m₄, k₁, k₂, k₃, and k₄ are independently 1, 2, or 3.

2. The method of claim 1, wherein R₂ is: 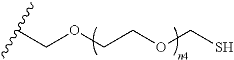
and n₄ is 45-55.

3. The method of claim 2, wherein m₁, m₂, and m₃ are each 1.

4. The method of claim 3, wherein n₁, n₂, and n₃ are independently 45-55.

5. The method of claim 3, wherein k₁, k₂, and k₃ are each 2.

6. The method of claim 1, wherein the PEG-thiol compound has the Formula:

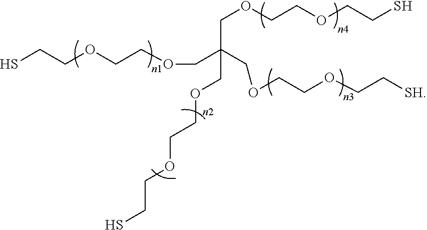

7. The method of claim 1, wherein the PEG-thiol compound is a PEG-10K-thiol.

8. The method of claim 1, wherein R₃ is: 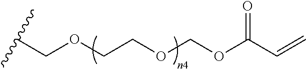
and n₄ is 45-55.

9. The method of claim 8, wherein m₁, m₂, and m₃ are each 1.

10. The method of claim 9, wherein n₁, n₂, and n₃ are independently 45-55.

11. The method of claim 9, wherein k₁, k₂, k₃, and k₄ are each 2.

12. The method of claim 1, wherein the PEG-acrylate compound has the Formula:

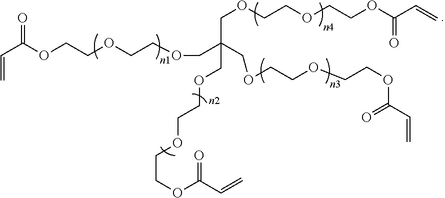

13. The method of claim 1, wherein the PEG-acrylate compound is a PEG-20K-acrylate.

14. The method of claim 1, wherein the composition further comprises a therapeutic agent selected from the group consisting of a corticosteroid, a decongestant, an immunotherapeutic agent, an adrenocorticoid, an analgesic agent, an analgesic adjunct, an analgesic-anesthetic, an anesthetic, an antibiotic, an antibacterial agent, an anti-infective agent, an antibiotic therapy adjunct, an antidote, an anti-emetic agent, an anti-fungal agent, an anti-inflammatory agent, an anti-vertigo agent, an anti-viral agent, a biological response modifier, a cytotoxic agent, a diagnostic aid, an immunizing agent, an immunomodulator, proteins, and peptides.

15. The method of claim 1, wherein the individual has undergone a surgical procedure.

16. The method of claim 1, wherein the individual has uncontrolled bleeding.

17. The method of claim 1, wherein the individual has a condition of the ear, nose, and/or throat selected from the group consisting of achalasia, acoustic neuroma/removal of acoustic neuroma, enlarged adenoids/adenoidectomy, infection, cancer, Meniere's disease, hay fever, allergies, asthma, sinusitis, diphtheria, and nasal polyps.

18. The method of claim 17, wherein the individual has a condition of the nose and the condition is selected from the group consisting of sinusitis, nasal polyps, nasal obstruction, rhinitis, bacterial infection, nosebleed, and nasal tumors.

19. The method of claim 18, wherein the individual has sinusitis and the sinusitis is acute sinusitis, recurrent sinusitis, or chronic sinusitis.

20. The method of claim 1, wherein the composition is configured to be applied as a liquid that hardens upon application to the nasal cavity.
